(19) United States
(12) Patent Application Publication
(10) Pub. No.: US 2024/0010922 A1
(43) Pub. Date: Jan. 11, 2024

(54) CATALYSTS AND PROCESSES FOR THE CONVERSION OF SYNTHESIS GAS TO LIQUEFIED PETROLEUM GAS (LPG) HYDROCARBONS

(71) Applicant: GTI Energy, Des Plaines, IL (US)

(72) Inventors: Patrick LITTLEWOOD, Chicago, IL (US); Terry MARKER, Park Ridge, IL (US); Michael BRADFORD, Des Plaines, IL (US)

(21) Appl. No.: 18/218,403

(22) Filed: Jul. 5, 2023

Related U.S. Application Data
(60) Provisional application No. 63/358,406, filed on Jul. 5, 2022.

(51) Int. Cl. C10G 2/00; C10L 3/12; B01J 29/70; B01J 29/74; B01J 29/72; B01J 29/06; B01J 29/80

(52) U.S. Cl. CPC C10G 2/334; C10L 3/12; C10G 2/333; B01J 29/7007; B01J 29/7415; B01J 29/7215; B01J 29/061; B01J 29/80; C10G 2400/28; C10G 2300/4018; C10G 2300/70; B01J 2229/183; B01J 2229/20; B01J 2229/26; B01J 2029/062

(57) ABSTRACT

Liquefied petroleum gas (LPG) synthesis catalyst systems are disclosed that provide activities for both alcohol (e.g., methanol) synthesis and in situ dehydration of the alcohol (e.g., methanol) to hydrocarbons, and particularly the LPG hydrocarbons propane and/or butane. The incorporation of a stabilizer such as platinum and/or yttrium (e.g., as yttria or yttrium oxide) can benefit these catalyst systems, particularly in terms of improving their activity and/or stability. Other advantages may be realized by the incorporation of promoters such as manganese (Mn), magnesium (Mg), and/or silicon (Si) into these catalyst systems, such as to improve selectivity to, and/or yield of, desired LPG hydrocarbons.

### CROSS-REFERENCE TO RELATED APPLICATIONS

This application claims priority to U.S. provisional application No. 63/358,406, filed Jul. 5, 2022, which is incorporated by reference in its entirety.

### FIELD OF THE INVENTION

Aspects of the invention relate to catalysts and associated processes for producing, from synthesis gas comprising H₂ and CO, products comprising propane and/or butane, for example those having a composition approximating that of liquefied petroleum gas (LPG). The propane and/or butane may have a substantial renewable carbon content.

### DESCRIPTION OF RELATED ART

The ongoing search for alternatives to crude oil, as a conventional source of carbon for hydrocarbon products, is increasingly driven by a number of factors. These include diminishing petroleum reserves, higher anticipated energy demands, and heightened concerns over greenhouse gas (GHG) emissions from sources of non-renewable carbon. Hydrocarbon products of greatest industrial significance and interest, in terms of having their carbon content replaced with non-petroleum derived carbon, include transportation and heating fuels as well as precursors for specialty chemicals. The particular hydrocarbons propane and/or butane are present in many of these products, a common example of which is liquefied petroleum gas (LPG).

A key commercial process for converting methane, biomass, coal, or other carbonaceous feedstocks into fuels involves a first conversion step to produce synthesis gas (syngas), followed by a second, downstream Fischer-Tropsch (FT) conversion step. With respect to the first conversion step, known processes for the production of syngas include partial oxidation reforming and autothermal reforming (ATR), based on the exothermic oxidation of methane with oxygen. Steam methane reforming (SMR), in contrast, uses steam as the oxidizing agent, such that the thermodynamics are significantly different, not only because the production of steam itself can require an energy investment, but also because reactions involving methane and water are endothermic. More recently, it has also been proposed to use carbon dioxide as the oxidizing agent for methane, such that the desired syngas is formed by the reaction of carbon in its most oxidized form (CO₂) with carbon in its most reduced form (CH₄). This reaction has been termed the “dry reforming” of methane, and because it is highly endothermic, thermodynamics for the dry reforming of methane are less favorable compared to ATR or even SMR. Gasification and pyrolysis have also been in extensive use for converting both renewable and non-renewable sources of carbon (e.g., biomass and coal) into syngas. A technology for processing diverse types of solid feedstocks including biomass, municipal solid waste, and plastics, which yields syngas in combination with deoxygenated hydrocarbon products suitable for use as gasoline and/or diesel fuel, is known as hydropyrolysis and is described in U.S. Pat. Nos. 8,492,600 and 10,619,105, as well as other patents assigned to Gas Technology Institute (Des Plaines, IL).

With respect to the second step involving FT conversion, synthesis gas containing a mixture of hydrogen and carbon monoxide (CO) is subjected to successive cleavage of C—O bonds and formation of C—C bonds with the incorporation of hydrogen. This mechanism provides for the formation of hydrocarbons, and particularly straight-chain alkanes with a distribution of molecular weights that can be controlled to some extent by varying the FT reaction conditions (temperature and feed H₂:CO ratio) and catalyst properties. Such properties include pore size and other characteristics of the support material. The choice of catalyst can impact FT product yields in other respects. For example, iron-based FT catalysts tend to produce more oxygenates, whereas ruthenium as the active metal tends to produce exclusively paraffins. The reaction pathways of FT synthesis follow a statistical kinetic model, which leads to hydrocarbons having an Anderson-Schultz-Flory distribution of their carbon numbers. In the case of targeting the C₃ and C₄ hydrocarbons, i.e., propane and butane, this generally involves operating in a low conversion regime with a significant co-production of methane and ethane. Higher conversions, on the other hand, generate C₅⁺ hydrocarbons that are liquid at room temperature. Other potential routes for the production of LPG hydrocarbons from syngas are described by K. Asami et al. (STUDIES IN SURFACE SCIENCE AND CATALYSIS 147 (2004) 427-432); Q. Zhang et al. (FUEL PROCESSING TECHNOLOGY85 (2004) 1139-1150); and Q. Ge et al. (JOURNAL OF MOLECULAR CATALYSIS A: CHEMICAL 278 (2007) 215-219.

In terms of known pathways offering potential conversion routes to LPG hydrocarbons from synthesis gas, which is desirably derived from renewable methane (e.g., present in biogas) or biomass, improvements are needed in a number of areas. These include reaction product selectivity and yield, in combination with catalyst stability, all of which parameters significantly impact commercial viability. The management of CO₂ that is often present in the synthesis gas, for example as a component of a gaseous feed mixture that is subjected to upstream reforming or otherwise as a component of a gasification or pyrolysis effluent, poses another challenge. Overall, the state of the art would benefit from technologies for the efficient conversion of industrially available sources of synthesis gas, whether obtained as a standalone feed or from an upstream processing stage (e.g., a reforming stage) of an integrated process, to products comprising propane and/or butane. Industrially relevant examples of such products are those having a composition approximating that of liquefied petroleum gas (LPG). With respect to the practical impact of such technologies, a current objective of a number of countries around the world is to reduce deforestation and the generation of pollution, both of which result from the burning of wood for heating and cooking. However, because of the remoteness of many locations and the associated, long transportation routes, petroleum-derived LPG is priced at a premium and therefore not considered a viable alternative to wood. Accordingly, a number of significant advantages could be gained by efficiently obtaining LPG hydrocarbons from renewable resources that provide synthesis gas. These advantages include freedom from the need to import petroleum-derived LPG, a reduction in GHG emissions, improvement in air quality, and the potential stimulation of local economies, particularly in poorer regions.

### SUMMARY OF THE INVENTION

Aspects of the invention are associated with the discovery of liquefied petroleum gas (LPG) synthesis catalyst systems that provide activities for both alcohol (e.g., methanol) synthesis and in situ dehydration of the alcohol (e.g., methanol) to hydrocarbons, and particularly the LPG hydrocarbons propane and/or butane. Advantageously, the incorporation of a stabilizer, such as a noble metal stabilizer (e.g., platinum) or a non-noble metal stabilizer (e.g., yttrium in the form of as yttria or yttrium oxide) can benefit these catalyst systems, particularly in terms of improving their stability and thereby reducing, or even eliminating, requirements for regeneration. For example, known processes for the conversion of methanol to olefinic hydrocarbons (methanol-to-olefins, or MTO, processes) using solid acid catalysts require continuous catalyst regeneration (CCR) to address the problem of rapid catalyst coking. Due to stability improvements associated with LPG synthesis catalyst systems described herein, in representative embodiments these catalyst systems may be utilized in a fixed bed configuration, or otherwise in an alternative bed configuration (e.g., as a fluidized bed), but without continuous catalyst regeneration. Other aspects relate to benefits associated with the incorporation of promoters such as manganese (Mn), magnesium (Mg), and/or silicon (Si) into these catalyst systems, such as to improve selectivity to, and/or yield of, desired LPG hydrocarbons. With respect to improvements associated with the use of stabilizer(s) and/or promoter(s), those skilled in the art will appreciate that even modest increases in catalyst stability, selectivity, and/or per-pass yield will generally translate to very significant economic benefits on a commercial scale. In addition to reduced requirements for catalyst regeneration, such benefits may be attributed, for example, to a decreased formation of undesired byproducts, including catalyst coke precursors, and/or reduced recycle gas requirements.

Representative LPG synthesis catalyst systems have activities for both (i) alcohol (e.g., methanol) synthesis and/or ether (e.g., dimethyl ether or DME) synthesis, together with (ii) dehydration. These catalyst systems may comprise two catalyst types (e.g., in a macroscopically uniform mixture of particles) or otherwise a bi-functional catalyst (e.g., having a macroscopically uniform, particle-to-particle composition) comprising two types of functional constituents. In the case of two catalyst types (e.g., separate compositions, each being in the form of separate particles), these may include both (i) an alcohol (e.g., methanol) synthesis catalyst and/or an ether (e.g., DME) synthesis catalyst, and (ii) a dehydration catalyst, the latter of which may alternatively be referred to as an alcohol to LPG hydrocarbon conversion (ATLPG) catalyst, such as in the case of a methanol to LPG hydrocarbon conversion (MTLPG) catalyst. For simplicity, the term ATLPG catalyst, such as in the case of an MTLPG catalyst, may be used to additionally, or more broadly, characterize, respectively, an ether to LPG hydrocarbon conversion catalyst, such as in the case of a DME to LPG hydrocarbon conversion catalyst, in view of the conversion of synthesis gas to LPG hydrocarbons possibly proceeding through a mechanism whereby an ether, such as in the case of DME, is produced alternatively to, or in combination with, an alcohol such as methanol. Separate catalyst types may be present in a given catalyst system (e.g., contained in an LPG synthesis reactor) in the form of a mixture, in the form of individual beds of one type or another (e.g., one or more beds of an alcohol synthesis catalyst alone, and/or one or more beds of a dehydration catalyst alone), or a combination thereof (e.g., one or more beds of a mixture, and/or one or more beds of alcohol synthesis catalyst alone and/or one or more beds of dehydration catalyst alone). In one embodiment, a bed of an alcohol (e.g., methanol) synthesis catalyst may precede (e.g., be positioned upstream of) a bed of a dehydration catalyst. In the case of a bi-functional catalyst, the functional constituents may include both an alcohol (e.g., methanol) synthesis-functional constituent and a dehydration-functional constituent, the latter of which may alternatively be referred to as an alcohol to LPG hydrocarbon conversion—(ATLPG-) functional constituent, such as in the case of a methanol to LPG hydrocarbon conversion—(MTLPG-) functional constituent. Analogous to the terms ATLPG catalyst and MTLPG catalyst, used in the case of two catalyst types, the term ATLPG-functional constituent, such as in the case of an MTLPG-functional constituent, may be used to additionally, or more broadly, characterize, respectively, an ether to LPG hydrocarbon conversion functional constituent, such as in the case of a DME to LPG hydrocarbon conversion functional constituent, in view of the conversion of synthesis gas to LPG hydrocarbons possibly proceeding through the production of an ether, such as DME.

According to preferred embodiments, LPG synthesis catalyst systems, in addition to comprising any of the general and specific alcohol (e.g., methanol) synthesis catalysts and dehydration catalysts described herein, or otherwise comprising any of the general and specific alcohol (e.g., methanol) synthesis-functional constituents and dehydration-functional constituents described herein, may further comprise a stabilizer, such as a noble metal stabilizer (e.g., platinum) or a non-noble metal stabilizer (e.g., yttrium (Y)) in its elemental form or in a compound form (e.g., in the form of yttria or yttrium oxide (Y₂O₃)). The stabilizer, such as platinum, yttrium, or other stabilizer as described herein may be present in a catalyst (e.g., an alcohol synthesis catalyst and/or dehydration catalyst as described herein) or may be present in a functional constituent (e.g., an alcohol synthesis-functional constituent or a dehydration-functional constituent as described herein). Alternatively, or in combination, the noble metal stabilizer (e.g., platinum) or non-noble metal stabilizer (e.g., yttrium) may be present in a separate composition of a catalyst system as described herein. For example, the catalyst system, any catalyst or functional constituent of the system, and/or any separate composition, may comprise a noble metal stabilizer (e.g., platinum) or non-noble metal stabilizer (e.g., yttrium in its elemental form, oxide form, or other form) in an amount as described herein.

According to other preferred embodiments, these systems, in addition to comprising any of the general and specific alcohol (e.g., methanol) synthesis catalysts and dehydration catalysts described herein, or otherwise comprising any of the general and specific alcohol (e.g., methanol) synthesis-functional constituents and dehydration-functional constituents described herein, may further comprise one or more promoters selected from the group consisting of manganese (Mn), magnesium (Mg), and silicon (Si), the promoter(s) being independently in elemental form or a compound form (e.g., oxide form). For example, representative catalyst systems may comprise such promoter(s) in addition to a noble metal stabilizer (e.g., platinum) or non-noble metal stabilizer (e.g., yttrium). The one or more promoters may be present in a catalyst (e.g., an alcohol synthesis catalyst and/or dehydration catalyst as described herein) or may be present in a functional constituent (e.g., an alcohol synthesis-functional constituent or a dehydration-functional constituent as described herein). Alternatively, or in combination, the one or more promoters may be present in a separate composition of a catalyst system as described herein. For example, the catalyst system, any catalyst or functional constituent of the system, and/or any separate composition, may comprise one or more promoters (e.g., independently in elemental forms, oxide forms, or other forms), independently in amounts, or otherwise in combined amounts, as described herein.

In a given catalyst system, the dehydration catalyst or the dehydration-functional constituent may comprise predominantly (e.g., greater than 50%), substantially all (e.g., greater than 95%), or all of the noble metal stabilizer (e.g., platinum) and/or non-noble metal stabilizer (e.g., yttrium) content of that system. However, according to alternative embodiments, beneficial effects may be obtained in a catalyst system in which the alcohol (e.g., methanol) synthesis catalyst or the alcohol (e.g., methanol) synthesis-functional constituent may comprise predominantly (e.g., greater than 50%), substantially all (e.g., greater than 95%), or all of the noble metal stabilizer (e.g., platinum) or non-noble metal stabilizer (e.g., yttrium) content of that system. In the case of noble metal stabilizer(s), the content of the stabilizer(s) may be based on the amount, or combined amount, of one or more of a noble metal selected from platinum (Pt), rhodium (Rh), ruthenium (Ru), palladium (Pd), silver (Ag), osmium (Os), iridium (Ir), and gold (Au). In the case of non-noble metal stabilizer(s), the content of the stabilizer(s) may be based on the amount, or combined amount, of one or more of a non-noble metal selected from Group 3 or Group 4 of the Periodic Table (e.g., the amount of yttrium) and/or one or more lanthanides. The same considerations, in terms of the distribution of a stabilizer (e.g., platinum and/or yttrium) between catalysts or functional constituents, apply independently to the distribution of one or more promoter(s) described herein. Accordingly, the alcohol (e.g., methanol) synthesis catalyst or the alcohol (e.g., methanol) synthesis-functional constituent may comprise predominantly (e.g., greater than 50%), substantially all (e.g., greater than 95%), or all of the content of promoter(s) of that system. Alternatively, the dehydration catalyst or the dehydration-functional constituent may comprise predominantly (e.g., greater than 50%), substantially all (e.g., greater than 95%), or all of the content of the promoter(s) of that system. The content of the promoter(s) may be based on the amount, or combined amount, of one or more of Mn, Mg, and Si, present in the system. In the case of either a catalyst mixture or a bi-functional catalyst, (i) the respective alcohol (e.g., methanol) synthesis catalyst or alcohol (e.g., methanol) synthesis-functional constituent may comprise one or more alcohol (e.g., methanol) synthesis-active metals selected from the group consisting of Cu, Zn, Al, Pt, Pd, and Cr, and/or (ii) the respective dehydration catalyst or dehydration-functional constituent may comprise a zeolite or non-zeolitic molecular sieve. In the case of such alcohol (e.g., methanol) synthesis-active metals being Pt and/or Pd, these may, in addition to being considered alcohol (e.g., methanol) synthesis-active metals, also be considered noble metal stabilizers. In the case of a dehydration catalyst or dehydration-functional constituent comprising a zeolite or non-zeolitic molecular sieve, the stabilizer(s) may be present in ion-exchange sites thereof, i.e., the dehydration catalyst or dehydration-functional constituent may comprise an ion-exchanged zeolite or an ion-exchanged non-zeolitic molecular sieve, having been prepared by ion-exchange to achieve a desired distribution of the stabilizer(s) within the zeolite or non-zeolitic molecular sieve.

Embodiments of the invention are directed to an LPG synthesis catalyst system comprising: (i) an alcohol (e.g., methanol) synthesis catalyst, and (ii) a dehydration catalyst (or ATLPG catalyst, such as an MTLPG catalyst), wherein the alcohol (e.g., methanol) synthesis catalyst and/or the dehydration catalyst comprises a stabilizer, such as a noble metal stabilizer (e.g., platinum) or a non-noble metal stabilizer such as yttrium (Y) in its elemental form or in a compound form. Other embodiments are directed to an LPG synthesis catalyst system comprising, as constituents of a bi-functional catalyst: (i) an alcohol (e.g., methanol) synthesis-functional constituent, and (ii) a dehydration-functional constituent (or ATLPG-functional constituent, such as an MTLPG-functional constituent), wherein the methanol synthesis-functional constituent and/or the dehydration-functional constituent comprises a stabilizer, such as a noble metal stabilizer (e.g., platinum) or a non-noble metal stabilizer such as yttrium (Y) in its elemental form or in a compound form. In the case of its elemental form, this may include its zerovalent atomic state (e.g., Pt⁰), or otherwise an ionic state (e.g., Pt⁺² or PC⁺³), with an excess or deficiency of electrons in the valance shell, such as in the cationic state in the case of having been incorporated into a support material by ion-exchange, as described herein. In any of the above embodiments, the stabilizer reduces deactivation of the dehydration catalyst or LPG synthesis catalyst system as a whole. Such reduction in deactivation may be measured experimentally in comparative testing of the dehydration catalyst or LPG synthesis catalyst, with and without the addition of the stabilizer. Yet other embodiments of the invention are directed to an LPG synthesis catalyst system comprising: (i) an alcohol (e.g., methanol) synthesis catalyst, and (ii) a dehydration catalyst (or ATLPG catalyst, such as an MTLPG catalyst), wherein the alcohol (e.g., methanol) synthesis catalyst and/or the dehydration catalyst comprises one or more promoters selected from the group consisting of manganese (Mn), magnesium (Mg), and silicon (Si), said promoter(s) being independently in elemental form or a compound form (e.g., oxide form). Still other embodiments are directed to an LPG synthesis catalyst system comprising, as constituents of a bi-functional catalyst: (i) an alcohol (e.g., methanol) synthesis-functional constituent, and (ii) a dehydration-functional constituent (or ATLPG-functional constituent, such as an MTLPG-functional constituent), wherein the alcohol (e.g., methanol) synthesis-functional constituent and/or the dehydration-functional constituent comprises one or more promoters selected from the group consisting of manganese (Mn), magnesium (Mg), and silicon (Si), said promoter(s) being independently in elemental form or a compound form (e.g., oxide form).

Further embodiments are directed to a process for producing an LPG product comprising propane and/or butane (and preferably both), the process comprising contacting a synthesis gas comprising H₂ and CO with an LPG synthesis catalyst system as described herein, and particularly such catalyst system comprising either separate catalysts or a bi-functional catalyst. In representative processes, the LPG synthesis catalyst systems described herein may be used to provide novel pathways for the production of liquefied petroleum gas (LPG) products comprising propane and/or butane, and in certain cases renewable LPG products, i.e., in which some or all (e.g., at least about 70%) of their carbon content (whether expressed on a wt-% or mole-% basis) is renewable carbon that is not derived from petroleum. Advantageously, whether or not the carbon content is renewable carbon, at least a portion (e.g., at least about 20%, at least about 30%, or at least about 40%) of the total carbon content of representative LPG products described herein may be derived from CO₂, for example being present as a component of a methane-containing gaseous feed mixture (e.g., biogas) that is subjected to upstream reforming or otherwise being present as a component of a gasification or pyrolysis effluent. In the case of a non-renewable carbon content that is derived from CO₂, such CO₂ may be obtained, for example, as a fossil fuel combustion product or a fossil fuel reforming product. In either case, it can be appreciated that CO₂ used to provide at least a portion of the total carbon content is beneficially utilized as LPG, rather than being directly released into the atmosphere. Within the environment of an LPG synthesis reactor containing the catalyst system, CO₂ may be present in an equilibrium or non-equilibrium amount, together with H₂, CO, and H₂O as other reactants/products of the reversible water-gas shift (WGS) reaction.

Yet other embodiments are directed to a dehydration catalyst (or ATLPG catalyst, such as an MTLPG catalyst) comprising a stabilizer, such as a noble metal stabilizer (e.g., platinum) or a non-noble metal stabilizer such as yttrium (Y) in its elemental form or in a compound form (e.g., its oxide form) on a solid acid support comprising a zeolite or non-zeolitic molecular sieve. The catalyst may further comprise one or more promoters selected from the group consisting of Mn, Mg, and Si, the one or more promoters being independently in their respective elemental form or a respective compound form (e.g., oxide form). In the case of any such stabilizer(s) and/or promoter(s), these may be present in ion-exchange sites of a zeolite or non-zeolitic molecular sieve, as a component of the dehydration catalyst, i.e., the dehydration catalyst may comprise an ion-exchanged zeolite or an ion-exchanged non-zeolitic molecular sieve, having been prepared by ion-exchange to achieve a desired distribution of the stabilizer(s) and/or promoter(s) within the zeolite or non-zeolitic molecular sieve.

These and other embodiments, aspects, and advantages relating to the present invention are apparent from the following Detailed Description.

### BRIEF DESCRIPTION OF THE DRAWINGS

A more complete understanding of the exemplary embodiments of the present invention and the advantages thereof may be acquired by referring to the following description in consideration of the accompanying figures.

FIG. 1 depicts the pore size distribution of zeolite support material, resulting from ion-exchange (IE) as a technique for preparing a dehydration catalyst or dehydration-functional constituent, and more particularly the preservation of the overall structure of this material, including its pore volume, before and after ion-exchange. This is compared with the por size distribution resulting from the alternative technique of incipient wetness impregnation (IWI).

FIG. 2 depicts a comparison of the performance, in terms of CO conversion, among LPG synthesis catalyst systems in which a zeolite support material, used in a dehydration catalyst, includes various amounts of a platinum stabilizer that has been added by ion-exchange.

FIG. 3 depicts the initial performance, up to about 50 days of operation, of LPG synthesis catalyst systems comprising zeolite beta as a component of a dehydration catalyst. In one of these catalyst systems, the zeolite beta did not have a Pt stabilizer (CATALYST 0), and in another of these catalysts systems, the dehydration catalyst was prepared from zeolite beta in powder form, without an alumina binder (CATALYST 5).

FIG. 4 depicts a comparison of the performance, in terms of the rate of non-selective methane production (in moles per minute per kilogram of catalyst), of LPG synthesis catalysts systems in which the Pt was incorporated into zeolite beta by ion-exchange (IE) and by incipient wetness impregnation (IWI).

FIG. 5 depicts a comparison of the performance, in terms of the rate of LPG hydrocarbon production (in moles per minute per kilogram of catalyst), of LPG synthesis catalysts systems in which the Pt was incorporated into zeolite beta by ion-exchange (IE) and by incipient wetness impregnation (IWI), for which the corresponding methane production rate is shown in FIG. 4.

FIG. 6 depicts a comparison of the performance, in terms of the percentage of CO conversion, of LPG synthesis catalysts systems in which the Pt was incorporated into zeolite beta by ion-exchange (IE) and by incipient wetness impregnation (IWI), for which the corresponding methane production rate is shown in FIG. 4 and the corresponding LPG hydrocarbon production rate is shown in FIG. 5.

FIG. 7 depicts a comparison of the performance, in terms of the percentage of LPG hydrocarbon yield, of LPG synthesis catalysts systems in which the Pt was incorporated into zeolite beta by ion-exchange (IE) and by incipient wetness impregnation (IWI), for which the corresponding methane production rate is shown in FIG. 4, the corresponding LPG hydrocarbon production rate is shown in FIG. 5, and the corresponding percentage of CO conversion is shown in FIG. 6.

### DETAILED DESCRIPTION

The expressions “wt-%” and “mol-%,” are used herein to designate weight percentages and molar percentages, respectively. The expressions “wt-ppm” and “mol-ppm” designate weight and molar parts per million, respectively. For ideal gases, “mol-%” and “mol-ppm” are equal to percentages by volume and parts per million by volume, respectively. In some cases, a percentage, “%,” is given with respect to values that are the same, whether expressed as a weight percentage or a molar percentage. For example, the percentage of the carbon content of the LPG product that is renewable carbon, has the same value, whether expressed as a weight percentage or a molar percentage.

The term “substantially,” as used herein, refers to an extent of at least 95%. For example, the phrase “substantially all” may be replaced by “at least 95%.”

A “synthesis gas comprising H₂ and CO,” or more simply “synthesis gas,” as described herein, may be representative of a portion of, or the entirety of, the material that is fed or input, e.g., that is input in one feed stream, or in two or more separate or combined feed streams, to an LPG synthesis reactor, used to carry out the conversion of at least a portion of the H₂ and CO to propane and/or butane that is contained in an LPG product. The synthesis gas comprising Hz and CO may be, or may comprise, in particular embodiments, a synthesis gas intermediate, or portion thereof, which is produced in an upstream reaction stage, such as a stage for carrying out reforming to generate the H₂ and CO. Whether or not obtained from a synthesis gas intermediate, at least a portion of the H₂ and CO in the synthesis gas may be converted by contact with an LPG synthesis catalyst system as described herein, to propane and/or butane that is contained in the LPG product. This conversion may proceed through a mechanism whereby an alcohol (e.g., methanol) produced from H₂ and CO (according to an alcohol synthesis reaction) is dehydrated to LPG hydrocarbons and water. In view of the hydrogen requirement for alcohol (e.g., methanol) synthesis and dehydration, the synthesis gas may have an H₂:CO molar ratio of at least about 2.0, such as from about 2.0 to about 2.5. Such molar ratios may be obtained, optionally following an adjustment (e.g., increase) occurring upstream of the conversion of the synthesis gas (e.g., upstream of an LPG synthesis reactor).

Alternatively to, or in combination with, alcohol synthesis, the conversion of synthesis gas to LPG hydrocarbons may proceed through a mechanism whereby an ether (e.g., DME) is produced. For example, in the case of a combination, methanol or other alcohol produced initially may be dehydrated to DME or other ether, which is then further dehydrated to LPG hydrocarbons. Accordingly, the terms “alcohol synthesis catalyst” and “alcohol synthesis-functional constituent” should be understood to refer to catalysts and functional constituents that may catalyze, or at least lead to (mechanistically), the formation of ethers (e.g., DME), alternatively to, or in combination with, the formation of alcohols (e.g., methanol). This is consistent with the term ATLPG catalyst, such as in the case of an MTLPG catalyst, being used, as described above, to additionally, or more broadly, characterize, respectively, an ether to LPG hydrocarbon conversion catalyst, such as in the case of a DME to LPG hydrocarbon conversion catalyst. This is also consistent with the term ATLPG-functional constituent, such as in the case of an MTLPG-functional constituent, being used, as described above, to additionally, or more broadly, characterize, respectively, an ether to LPG hydrocarbon conversion functional constituent, such as in the case of a DME to LPG hydrocarbon conversion functional constituent.

Any source of synthesis gas comprising H₂ and CO may be used as a feed to an LPG synthesis reactor, in representative LPG synthesis processes, including a synthesis gas that is produced at least partly by reforming. The synthesis gas may comprise H₂ and CO in any suitable amounts (concentrations), such as in combined amount of greater than about 25 mol-% (e.g., from about 25 mol-% to 100 mol-%), greater than about 50 mol-% (e.g., from about 50 mol-% to about 99 mol-%), or greater than about 75 mol-% (e.g., from about 75 mol-% to about 99 mol-%). With respect to any such combined amounts (concentrations), the H₂:CO molar ratio of the synthesis gas may be may be from about 1.0 to about 7.0, such as from about 4.0 to about 6.5, in the case of relatively high ratios. Otherwise, in the case of relatively low ratios, the H₂:CO molar ratio of the synthesis gas intermediate may be from about 1.0 to about 3.0, such as from about 1.8 to about 2.4. The LPG product, comprising propane (C₃H₈) and/or butane (CLEO, may be obtained using catalyst systems as described herein for catalyzing reactions of methanol synthesis and dehydration, as follows:

14H₂+7CO→7CH₃OH(methanol synthesis), and

7CH₃OH+2H₂→C₃H₈+C₄H₁₀+7H₂O(dehydration).

According to the above reactions, the LPG hydrocarbons propane and butane may be produced from synthesis gas through a methanol intermediate. As noted above, LPG hydrocarbons may also be produced from synthesis gas through a DME intermediate, such as in the case of Hz and CO reacting to form DME (CH₃OCH₃) and water, followed by dehydration of DME to LPG hydrocarbons. Otherwise, LPG hydrocarbons may be produced from synthesis gas through both a methanol intermediate and a DME intermediate, such as in the case of H₂ and CO reacting to form methanol (CH₃OH), followed by dehydration of methanol to DME, and further dehydration of DME to LPG hydrocarbons.

Alternatively, or in combination, CO₂ present in the synthesis gas may likewise advantageously be reacted in the initial methanol synthesis, according to a second pathway. For example, in the case of producing the same number of moles of CH₃OH shown in the first reaction above, CO₂, rather than CO, may be consumed according to:

21H₂+7CO₂→7CH₃OH+7H₂O(methanol synthesis).

In view of any of these proposed routes to LPG hydrocarbons, the synthesis gas may have an H₂:CO molar ratio of at least about 1.0 (e.g., from about 1.0 to about 3.5 or from about 1.5 to about 3.0), or more preferably at least about 2.0 (e.g., from about 2.0 to about 4.0, from about 2.0 to about 3.0, or from about 2.0 to about 2.5). In some cases, excess H₂ (i.e., H₂ in excess of the stoichiometric amount needed to react with CO and/or CO₂ to form a methanol intermediate according to the reactions above, or otherwise a DME intermediate) may be desired to improve stability of a given LPG synthesis catalyst system.

More generally, the LPG product comprising propane and/or butane may be produced through synthesis of a methanol intermediate or higher alcohol intermediate, obtained from the reaction of H₂ with CO or CO₂, according to the following generalized reactions:

2n H₂+n CO→CₙH₂ₙ₊₁OH+(n−1)H₂O and/or

(3n+b)H₂+(n+b)CO₂→CₙH₂ₙ₊₁OH+(2n+b−1)H₂O+bCO(alcohol synthesis), and

(7/n)CₙH₂ₙ₊₁OH+2H₂→C₃H₈+C₄H₁₀+(7/n)H₂O(dehydration).

According to these reactions, the LPG hydrocarbons propane and butane may be produced from synthesis gas, more generally through an alcohol intermediate. As noted above, LPG hydrocarbons may also be produced from synthesis gas generally through an ether intermediate, such as in the case of H₂ and CO reacting to form an ether (e.g., CₙH₂ₙ₊₁O CₙH₂ₙ₊₁) and water, followed by dehydration of the ether to LPG hydrocarbons. Otherwise, LPG hydrocarbons may be produced from synthesis gas through both an alcohol intermediate and an ether intermediate, such as in the case of H₂ and CO reacting to form an alcohol, followed by dehydration of the alcohol to the ether, and further dehydration of the ether to LPG hydrocarbons.

Independently of, or in combination with, the representative amounts (concentrations) of H₂ and CO above and/or representative H₂:CO molar ratios above, the synthesis gas may further comprise CO₂, for example in an amount of at least about 5 mol-% (e.g., from about 5 mol-% to about 50 mol-%), at least about 10 mol-% (e.g., from about 10 mol-% to about 35 mol-%), or at least about 15 mol-% (e.g., from about 15 mol-% to about 30 mol-%). In such cases, the balance of the synthesis gas may be, or may substantially be, H₂ and CO in combination, for example in an H₂:CO molar ratio as described herein.

In the processing of a synthesis gas comprising H₂ and CO, catalyst systems as described herein can provide important advantages in terms of activity and/or stability, leading to process economics favorable for commercialization. More specifically, with respect to alcohol (e.g., methanol) synthesis catalysts, dehydration catalysts, and bi-functional catalysts as described herein, these catalysts, as is the case with catalysts generally, deactivate over time. A significant contributing factor to catalyst deactivation, with respect to reactions involving the conversion of synthesis gas to LPG hydrocarbons, is coking caused by the formation of larger organic byproduct molecules (e.g., polyaromatic compounds), which can essentially block catalyst pores and/or serve as precursors for even higher molecular weight species that deposit on catalyst surfaces as coke. In this regard, the presence of the byproduct formaldehyde in the environment of alcohol (e.g., methanol) synthesis catalysts, dehydration catalysts, and bi-functional catalysts as described herein, or elsewhere in an overall process utilizing a step of converting synthesis gas to LPG hydrocarbons, may be detrimental. For example, under conditions of LPG synthesis, formaldehyde is believed to terminate chain growth reactions that produce LPG hydrocarbons and instead lead to the formation of the polyaromatic compounds having the noted detrimental effects. In the face of the various deactivation mechanisms, it has been surprisingly discovered that the addition of a noble metal (e.g., one or more of Pt, Rh, Ru, Pd, Ag, Os, Ir, and Au) and/or a non-noble metal (e.g., a metal selected from Group 3 or Group 4 of the Periodic Table, or a lanthanide) to the catalyst system can improve its activity and/or stability. For example, the addition of a stabilizer may increase the activity of an alcohol (e.g., methanol) synthesis catalyst, a dehydration catalyst, and/or a bi-functional catalyst as described herein, and/or may increase the activity of the catalyst system as a whole. The addition of a stabilizer may alternatively, or in combination, reduce the deactivation (or deactivation rate) of an alcohol (e.g., methanol) synthesis catalyst, a dehydration catalyst, and/or a bi-functional catalyst as described herein, and/or may reduce the deactivation (or deactivation rate) of the catalyst system as a whole. In this regard, the term “stabilizer,” as described herein, therefore extends to metal additives, in their elemental form or in a compound form, which may generally have the effect of increasing activity and/or reducing deactivation.

In terms of increasing activity and/or reducing deactivation, one or both of these beneficial characteristics of a stabilizer may be demonstrated by comparative performance tests of LPG synthesis catalyst systems, including a given alcohol (e.g., methanol) synthesis catalyst, dehydration catalyst, or bi-functional catalyst used in a given system, which are the same in all respects except for the presence of the stabilizer(s) in one catalyst system and the absence of the same stabilizer(s) in another. Such comparative performance tests, of a process for producing an LPG product, use a standard set of conditions (e.g., pressure, temperature, and space velocity) that are preferably characteristic of LPG synthesis reaction conditions as described herein, and such conditions are more particularly used for processing a synthesis gas having a standard composition (e.g., 67 mol-% H₂ and 33 mol-% CO). An activity increase may be evidenced by a higher degree of conversion of the synthesis gas at a given catalyst temperature (or average bed temperature), or alternatively by a lower catalyst temperature (or average bed temperature) needed to achieve a given level of conversion. A reduction in deactivation, or stability increase, may be evidenced by a lower rate of decrease in a given performance parameter (e.g., conversion and/or LPG hydrocarbon yield) over tip e, while maintaining the conditions at constant values. Alternatively, a reduction in deactivation, or stability increase, may be evidenced by a lower rate of increase in severity of the conditions (e.g., temperature increase) over time, needed to maintain a given performance parameter conversion and/or LPG hydrocarbon yield). A reduction in deactivation, or stability increase, can manifest in less stringent requirements for catalyst regeneration, such as by prolonging on-stream catalyst utilization, between regeneration intervals; decreasing severity in regeneration conditions and/or end-of-life operating conditions; and/or enabling the use of simpler catalyst bed configurations, such as a fixed bed, allowing for periodic as opposed to continuous regeneration requirements. In the case of demonstrating an increase in activity and/or reduction in deactivation with respect to a dehydration catalyst in particular, and in the absence of a methanol synthesis catalyst, a suitable comparative performance test may involve, rather than processing a synthesis gas having a standard composition, processing an alcohol-containing (e.g., methanol-containing) feed and/or ether-containing (e.g., DME-containing) feed having a standard composition, with improvements in process parameters being evidenced as described above.

According to certain representative embodiments, one or more stabilizers of a given LPG synthesis catalyst system may be characterized as a noble metal stabilizer (e.g., Pt, Rh, Ru, Pd, Ag, Os, Ir, and Au) or a non-noble metal stabilizer (e.g., a metal of Group 3 or Group 4 of the Periodic Table, or a lanthanide), any of which stabilizer(s) may be independently present in the catalyst system its/their elemental form or a compound form. For example, any noble metal stabilizer(s) may be present in its/their elemental form(s), and/or any non-noble metal stabilizer(s) may be present in its/their compound form(s) (e.g., an oxide form or carbonate form). A preferred noble metal stabilizer is platinum (Pt), and a preferred non-noble metal stabilizer is yttrium (Y). Aspects of the invention therefore relate to the use of one or more of these stabilizers in any of the catalyst systems described herein, for example as a component of a methanol synthesis catalyst, a dehydration catalyst, or a bi-functional catalyst, or otherwise as a separate composition, or component of a separate composition, of the catalyst system. Representative stabilizers may include one or more noble metals (e.g., one or more of Pt, Rh, Ru, Pd, Ag, Os, Ir, and Au) and/or one or more non-noble metals (e.g., metals selected from Group 3 or Group 4 of the Periodic Table, or lanthanides). Without being bound by any particular theory as to advantages that may be gained from the use of stabilizer(s), according to some embodiments such stabilizer(s) is/are believed to have beneficial activity in terms of suppressing the formation of coke precursors, and therefore the formation of coke itself, for example by selectively decomposing formaldehyde that may form/accumulate in, or otherwise be present in, the environment of the LPG synthesis reactor.

Accordingly, in some embodiments, any of the catalyst systems described herein may comprise a stabilizer such as a noble metal (e.g., platinum) or a non-noble metal (e.g., yttrium) in elemental form or in compound form, such as an oxide form or carbonate form. In the case of yttrium as a stabilizer, for example, this may be present in the form of yttria (yttrium oxide). Any stabilizer described herein, such as platinum or yttrium (e.g., in elemental form, or in an oxide form, such as in the case of yttria, or other form) may be a component of an alcohol synthesis catalyst (e.g., a methanol synthesis catalyst) or of an alcohol synthesis-functional constituent (e.g., a methanol synthesis-functional constituent) as described herein, or otherwise may be a component of a dehydration catalyst or of a dehydration-functional constituent as described herein. With respect to any of the catalyst systems described herein, one or more stabilizers (e.g., in elemental form, in the form of an oxide such as yttria or a carbonate or other form) may be present as a component of a catalyst or functional constituent, or otherwise as a separate composition, or component of a separate composition, of the catalyst system, in an amount, or a combined amount, from about 0.01 wt-% to about 10 wt-%, such as from about 0.05 wt-% to about 5 wt-% or from about 0.1 wt-% to about 1 wt-%, based on the weight of the stabilizer(s) (e.g., platinum or yttrium) relative to the weight of the catalyst system. In some embodiments, the weight of the catalyst system may be the combined weight of (i) an alcohol synthesis catalyst such as a methanol synthesis catalyst and (ii) a dehydration catalyst, or otherwise the weight of a bi-functional catalyst comprising (i) an alcohol-functional constituent such as a methanol-functional constituent, and (ii) a dehydration-functional constituent. In embodiments of (i) and (ii) being either catalysts or functional constituents, the weight of the catalyst system may also include any additional composition(s) of the catalyst system, such as a separate composition of the catalyst system that comprises any one or more stabilizers described above, such as platinum or yttrium. Based on the weight of the stabilizer(s) (e.g., platinum or yttrium), and, relative to the weight of the alcohol synthesis catalyst alone, the dehydration catalyst alone, the alcohol synthesis-functional constituent alone, or the dehydration-functional constituent alone, the stabilizer(s) such as platinum or yttrium (e.g., in elemental form, in the form of an oxide such as yttria or a carbonate or other form) may be present in an amount from about 0.03 wt-% to about 15 wt-%, such as from about 0.08 wt-% to about 8 wt-% or from about 0.2 wt-% to about 2 wt-%. In preferred embodiments, the stabilizer(s) such as platinum or yttrium (e.g., in elemental form, in the form of an oxide such as yttria or a carbonate or other form) may be a component of the dehydration catalyst or dehydration-functional constituent, i.e., this catalyst or functional constituent comprises the stabilizer(s) such as platinum or yttrium (e.g., in elemental form, in the form of an oxide such as yttria or a carbonate or other form), such as in an amount described above. It is also possible for the alcohol synthesis catalyst or alcohol synthesis-functional constituent to comprise the stabilizer(s) such as platinum or yttrium (e.g., in elemental form, in the form of an oxide such as yttria or a carbonate or other form), for example in these amounts.

In view of further catalytic performance advantages that may be gained, optionally in combination with the use of one or more stabilizers such as platinum and/or yttrium, catalyst systems described herein may comprise one or more promoters selected from the group consisting of manganese (Mn), magnesium (Mg), and silicon (Si), with the one or more promoters being independently in elemental form or a compound form. Such performance advantages may reside in improvements in activity, selectivity, and/or yield. For example, manganese oxide (MnO₂), magnesium oxide (MgO), and/or silica (SiO₂), or other forms of Mg, Mg, and/or Si, may be component(s) of an alcohol synthesis catalyst, such as a methanol synthesis catalyst, or of an alcohol synthesis-functional constituent, such as a methanol synthesis-functional constituent as described herein, or otherwise may be component(s) of a dehydration catalyst or of a dehydration-functional constituent as described herein. With respect to any of the catalyst systems described herein, such promoters (e.g., independently in elemental forms, oxide forms, or other forms) may be present as a component of a catalyst or functional constituent, or otherwise as a separate composition or component of a separate composition, of the catalyst system. For example, any promoter, or combination of two or more of such promoters, may be present in an amount, or combined amount, from about 0.05 wt-% to about 12 wt-%, such as from about 0.1 wt-% to about 10 wt-% or from about 0.5 wt-% to about 8 wt-%, based on the weight of Mn, Mg, and/or Si relative to the weight of the catalyst system. In some embodiments, the weight of the catalyst system may be the combined weight of (i) an alcohol synthesis catalyst such as a methanol synthesis catalyst and (ii) a dehydration catalyst, or otherwise the weight of a bi-functional catalyst comprising (i) an alcohol synthesis-functional constituent such as a methanol synthesis-functional constituent, and (ii) a dehydration-functional constituent. In embodiments of (i) and (ii) being either catalysts or functional constituents, the weight of the catalyst system may also include any additional composition(s) of the catalyst system, such as a separate composition of the catalyst system that comprises one or more promoters. Based on the weight of Mn, Mg, and/or Si, and, relative to the weight of the alcohol synthesis catalyst alone, the dehydration catalyst alone, the alcohol synthesis-functional constituent alone, or the dehydration-functional constituent alone, the promoter(s) (e.g., independently in elemental forms, oxide forms, or other forms) may be present in an amount from about 0.08 wt-% to about 15 wt-%, such as from about 0.2 wt-% to about 12 wt-% or from about 0.8 wt-% to about 10 wt-%. In preferred embodiments, the promoter(s) (e.g., independently in elemental forms, oxide forms, or other forms) is/are component(s) of the alcohol synthesis catalyst (e.g., methanol synthesis catalyst) or alcohol synthesis-functional constituent (e.g., methanol synthesis-functional constituent), i.e., this catalyst or functional constituent comprises one or more promoter(s) selected from the group consisting of Mn, Mg, and Si (e.g., independently in elemental forms, oxide forms, or other forms), such as in an amount described above. It is also possible for the dehydration catalyst or dehydration-functional constituent to comprise such one or more promoter(s) (e.g., independently in elemental forms, oxide forms, or other forms), for example in these amounts.

Improvements in activity, selectivity, and/or yield due to the presence of one or more promoters may, as in the case of an improvement in stability as described above, be demonstrated by comparative performance tests of LPG synthesis catalyst systems that are the same in all respects except for the presence of one or more promoters in one catalyst system and the absence of the same such promoter(s) in another. Such comparative performance tests use a standard set of conditions (e.g., pressure, temperature, and space velocity) for processing a synthesis gas having a standard composition (e.g., 67 mol-% H₂ and 33 mol-% CO). An activity increase may be evidenced as described above with respect to determining this effect due to the use of a stabilizer. Selectivity and/or yield increases may be evidenced by comparative analysis of the compositions of LPG products obtained, for example by determining the proportion of converted carbon that forms propane and/or butane (a measure of selectivity) and/or by determining the overall amount of carbon input to the LPG synthesis catalyst system that forms propane and/or butane (a measure of yield). Improvements in activity, selectivity, and/or yield can manifest in reduced downstream separation and recycle requirements, thereby lowering operating costs.

An LPG synthesis catalyst system may comprise two or more different catalyst types, or a single catalyst having two or more different types of functional constituents. The different catalyst types or single catalyst may be contained in one or more LPG synthesis reactors (e.g., in a series or parallel arrangement), at least one of which is fed a synthesis gas comprising H₂ and CO, for contacting with the LPG synthesis catalyst system, or at least one catalyst type of the system. Preferably, the different catalyst types or single catalyst are contained within a single LPG synthesis reactor, but it is also possible, for example, for separate LPG synthesis reactors to contain each of the different catalyst types. It is also possible for separate LPG synthesis reactors to contain the different catalyst types at different weight ratios and/or in different bed configurations. In one embodiment, a first (upstream) LPG synthesis reactor (e.g., a methanol synthesis reactor) may contain an alcohol synthesis catalyst (e.g., methanol synthesis catalyst) as described herein, and a second (downstream) LPG synthesis reactor (e.g., a dehydration reactor) may contain a dehydration catalyst as described herein. The use of separate reactors allows for reaction conditions to be more precisely aligned with different stages of reactions used to carry out the synthesis of LPG hydrocarbons from a synthesis gas. In general, different catalyst types or a single catalyst may be utilized in any particular bed configuration (e.g., fixed bed or fluidized bed), or, in the case of different catalyst types in a fixed bed configuration, in any particular arrangement of individual beds of one catalyst type or another, such as in the case of using one or more beds an alcohol synthesis catalyst (e.g., methanol synthesis catalyst) alone, one or more beds of a dehydration catalyst alone, one or more beds of a mixture of catalyst types at a selected mixing ratio or differing mixing ratios, and/or combinations of such beds. A given LPG synthesis catalyst system (e.g., in the case of a fluidized bed configuration) may be operated with either continuous catalyst replacement (e.g., 0.05 wt-% to 0.5 wt-% per day via a slip stream) or continuous catalyst regeneration (e.g., of a similar magnitude of regenerated catalyst). Such replacement and/or regeneration may likewise be implemented with a moving bed configuration. Regardless of the particular bed configuration or particular arrangement of individual beds, preferably the catalyst types or single catalyst is/are in the form of discreet particles, as opposed to a monolithic form of catalyst. For example, such discreet particles of an alcohol synthesis catalyst e.g., methanol synthesis catalyst), a dehydration catalyst, or a bi-functional catalyst may have a spherical or cylindrical diameter of less than about 10 mm and often less than about 5 mm (e.g., about 2 mm). In the case of cylindrical catalyst particles (e.g., formed by extrusion), these may have a comparable length dimension (e.g., from about 1 mm to about 10 mm, such as about 5 mm).

LPG synthesis catalyst systems may, more particularly, comprise at least two components having different catalytic activities, with such components either being (a) separate compositions (e.g., each composition being in the form of separate particles) of an alcohol synthesis catalyst (e.g., a methanol synthesis catalyst) and a dehydration catalyst, or (b) functional constituents of a bi-functional catalyst (e.g., the catalyst being in the form of separate particles) that is a single composition having both an alcohol synthesis-functional constituent (e.g., a methanol synthesis-functional constituent) and a dehydration-functional constituent. As noted above, a dehydration catalyst may alternatively be referred to as an alcohol to LPG hydrocarbon conversion (ATLPG) catalyst, such as a methanol to LPG hydrocarbon conversion (MTLPG) catalyst, and a dehydration-functional constituent may alternatively be referred to as an alcohol to LPG hydrocarbon conversion—(ATLPG-) functional constituent, such as a methanol to LPG hydrocarbon conversion—(MTLPG-) functional constituent, with such terms having the meanings as described above and not precluding reaction mechanisms involving intermediate ether (e.g., DME) production alternatively to, or in combination with, intermediate alcohol (e.g., methanol) production.

The separate catalyst compositions, or otherwise the functional constituents of a bi-functional catalyst, may be present in equal or substantially equal weight ratios. For example, the (i) alcohol synthesis catalyst (e.g., methanol synthesis catalyst) and (ii) dehydration catalyst may be present in the catalyst mixture in a weight ratio of (i):(ii) of about 1:1. Otherwise, the (i) alcohol synthesis-functional constituent (e.g., methanol synthesis-functional constituent) and (ii) dehydration-functional constituent may be present in the bi-functional catalyst in a weight ratio of (i):(ii) of about 1:1. Generally, however, these weight ratios may vary, for example the weight ratios of (i):(ii) in each case may be from about 10:1 to about 1:10, such as from about 5:1 to about 1:5, or from about 3:1 to about 1:3.

In addition to such separate compositions of catalysts or single composition of a bi-functional catalyst, representative LPG synthesis catalyst systems may further comprise additional components, e.g., particles of silica or sand, acting to absorb heat and/or alter the distribution of solids. Such additional components may be present in an amount, for example, of at least about wt-% (e.g., from about 10 wt-% to about 80 wt-%), at least about 20 wt-% (e.g., from about wt-% to about 70 wt-%), or at least about 40 wt-% (e.g., from about 40 wt-% to about 60 wt-%), of a given catalyst system. Such additional components may therefore substantially lack catalytic activity and serve non-catalytic purposes. Alternatively, or in combination, additional components may include additional compositions having catalytic activity and/or additional functional constituents having catalytic activity. For example, representative LPG synthesis catalyst systems may comprise additional compositions as described above, such as an additional composition comprising a stabilizer such as platinum or yttrium (e.g., in elemental form, in the form of an oxide such as yttria or other form) and/or an additional composition comprising one or more promoters selected from the group consisting of Mn, Mg, and Si (e.g., independently in elemental forms, oxide forms, or other forms). In this regard, it can be appreciated that a catalyst system comprising an alcohol synthesis catalyst such as a methanol synthesis catalyst and a dehydration catalyst is not meant to preclude the presence of other catalysts. Likewise, the term “bi-functional catalyst” is not meant to preclude the presence of additional functional constituents. In some embodiments, however, an LPG synthesis catalyst system may consist of, or consist essentially of, two different catalyst types, or otherwise a single catalyst of such catalyst system may consist of, or consist essentially of, two different types of functional constituents. An LPG synthesis catalyst system may also consist of, or consist essentially of, a single type of bi-functional catalyst.

A representative alcohol synthesis catalyst (e.g., methanol synthesis catalyst) or alcohol synthesis-functional constituent (e.g., methanol synthesis-functional constituent) of a bi-functional catalyst may comprise one or more alcohol synthesis-active metals (e.g., methanol synthesis-active metals), with representative metals being selected from the group consisting of copper (Cu), zinc (Zn), aluminum (Al), platinum (Pt), palladium (Pd), and chromium (Cr). In the case of such alcohol (e.g., methanol) synthesis-active metals being Pt and/or Pd, these may, in addition to being considered alcohol (e.g., methanol) synthesis-active metals, also be considered noble metal stabilizers. Any alcohol synthesis-active metals may be in their elemental forms or compound forms. For example, in the case of Cu, Pt, and Pd, these metals are preferably in their elemental forms and, in the case of Zn, Al, and Cr, these metals are preferably in their oxide forms, namely ZnO, Al₂O₃, and Cr₂O₃, respectively. In some preferred embodiments, all or a portion of Cu, in case of an alcohol synthesis catalyst (e.g., a methanol synthesis catalyst) or alcohol synthesis-functional constituent (e.g., methanol synthesis-functional constituent) comprising this metal, may be in its oxide form CuO. A particular representative alcohol synthesis catalyst, which may more particularly be a methanol synthesis catalyst, is a copper and zinc oxide on alumina catalyst, comprising or consisting essentially of Cu/ZnO/Al₂O₃. Such “CZA” alcohol synthesis catalyst (e.g., methanol synthesis catalyst) may also be an alcohol synthesis-functional constituent (e.g., methanol synthesis-functional constituent) of a bi-functional catalyst.

In the case of an alcohol synthesis catalyst (e.g., methanol synthesis catalyst) or alcohol synthesis-functional constituent (e.g., methanol synthesis-functional constituent) of a bi-functional catalyst, the alcohol synthesis-active metals (e.g., methanol synthesis-active metals) Cu, Zn, Pt, Pd, and/or Cr, particularly when in their elemental forms, may be supported on a solid support. Representative solid supports comprise one or more metal oxides, for example those selected from the group consisting of aluminum oxide, silicon oxide, titanium oxide, zirconium oxide, magnesium oxide, calcium oxide, iron oxide, vanadium oxide, chromium oxide, nickel oxide, tungsten oxide, and strontium oxide. The phrase “on a solid support” is intended to encompass alcohol synthesis catalyst solid supports (e.g., methanol synthesis catalyst solid supports) and bi-functional catalyst solid supports in which the alcohol synthesis-active metal(s) (e.g., methanol synthesis-active metal(s)) is/are on the support surface and/or within a porous internal structure of the support. Specific examples of alcohol synthesis catalysts, such as methanol synthesis catalysts, or alcohol synthesis-functional constituents, such as methanol synthesis-functional constituents, therefore include Pd that is supported on a solid support of a metal oxide (e.g., aluminum oxide) and present in the catalyst or constituent in an amount as described herein.

For an alcohol synthesis catalyst (e.g., methanol synthesis catalyst) or an alcohol synthesis-functional constituent (e.g., methanol synthesis-functional constituent) comprising one or more of Cu, Zn, Al, Pt, Pd, and Cr, regardless of their particular form(s), such metal(s) may be present independently in an amount, in the respective alcohol synthesis catalyst (e.g., methanol synthesis catalyst) or bi-functional catalyst, generally from about 0.5 wt-% to about 45 wt-%, typically from about 1 wt-% to about 20 wt-%, and often from about 1 wt-% to about 10 wt-%, relative to the weight of the alcohol synthesis catalyst alone or the alcohol synthesis-functional constituent alone, or possibly relative to a bi-functional catalyst as a whole. In some embodiments, the metal Cu may be present, in an alcohol synthesis catalyst or bi-functional catalyst, in an amount from about 1 wt-% to about 25 wt-%, such as from about 1 wt-% to about 15 wt-%, relative to the weight of the alcohol synthesis catalyst alone or the alcohol synthesis-functional constituent alone, or possibly relative to a bi-functional catalyst as a whole. Independently or in combination with such amounts of Cu, the metal Zn may be present, in an alcohol synthesis catalyst such as a methanol synthesis catalyst, or bi-functional catalyst, in an amount from about 1 wt-% to about 20 wt-%, such as from about 1 wt-% to about 10 wt-%, relative to the weight of the alcohol synthesis catalyst alone or the alcohol synthesis-functional constituent alone, or possibly relative to a bi-functional catalyst as a whole. Independently or in combination with such amounts of Cu and/or Zn, the metal Al may be present, in an alcohol synthesis catalyst such as a methanol synthesis catalyst, or bi-functional catalyst, in an amount from about 1 wt-% to about 30 wt-%, such as from about 5 wt-% to about 20 wt-%, relative to the weight of the alcohol synthesis catalyst alone or the alcohol synthesis-functional constituent alone, or possibly relative to a bi-functional catalyst as a whole. Independently or in combination with such amounts of Cu, Zn, and/or Al, any one or more of the metals Pt, Pd, and/or Cr may be present, in an alcohol synthesis catalyst (e.g., methanol synthesis catalyst) or bi-functional catalyst, independently in an amount, or in a combined amount, from about 0.5 wt-% to about 10 wt-%, such as from about 1 wt-% to about 5 wt-%, relative to the weight of the alcohol synthesis catalyst alone or the alcohol synthesis-functional constituent alone, or possibly relative to a bi-functional catalyst as a whole.

The alcohol synthesis catalyst (e.g., methanol synthesis catalyst) or a methanol synthesis-functional constituent may further comprise a noble metal stabilizer (e.g., platinum) and/or non-noble metal stabilizer such as yttrium (e.g., in elemental form, in the form of an oxide such as yttria or other form) and/or one or more promoters selected from the group consisting of Mn, Mg, and/or Si (e.g., independently in elemental forms, oxide forms, or other forms), in respective amounts as described above. In the case of an alcohol synthesis catalyst (e.g., methanol synthesis catalyst) or alcohol synthesis-functional constituent (e.g., methanol synthesis-functional constituent) of a bi-functional catalyst, the alcohol synthesis-active metal(s) (e.g., methanol synthesis-active metal(s)), or any forms of such metals (e.g., their respective oxide forms), and optionally any solid support, may constitute all or substantially all of the catalyst or functional constituent. For example, the alcohol synthesis-active metal(s) (e.g., methanol synthesis-active metal(s)), or any forms of such metals, and optionally any solid support, may be present in a combined amount representing at least about 90%, at least about 95%, or at least about 99%, of the total weight of the alcohol synthesis catalyst (e.g., methanol synthesis catalyst) or alcohol synthesis-functional constituent (e.g., methanol synthesis-functional constituent). In the case of an alcohol synthesis catalyst or an alcohol synthesis-functional constituent further comprising one or more stabilizers and/or one or more promoters of Mn, Mg, and/or Si, the alcohol synthesis-active metal(s), or any forms of such metals, and optionally any solid support, together with the stabilizer(s) or any forms of the stabilizer(s) (e.g., platinum and/or yttrium in any form) and/or the promoter(s) or any forms of the promoter(s), may be present in a combined amount representing at least about 90%, at least about 95%, or at least about 99%, of the total weight of the alcohol synthesis catalyst (e.g., methanol synthesis catalyst) or alcohol synthesis-functional constituent (e.g., methanol synthesis-functional constituent).

In a representative alcohol synthesis catalyst (e.g., methanol synthesis catalyst) or bi-functional catalyst, any metal(s) other than Cu, Zn, Al, Pt, Pd, and/or Cr may be present in minor amounts, may be substantially absent, or may be absent. For example, any such other metal(s) may be independently present in an amount of less than about 1 wt-%, less than about 0.1 wt-%, or even less than about 0.05 wt-%, based on the total catalyst weight. Alternatively, any two or more of such other metals may be present in a combined amount of less than about 2 wt-%, less than about 0.5 wt-%, or even less than about 0.1 wt-%, based on the total catalyst weight. According to particular embodiments, for example in the case of (i) an alcohol synthesis catalyst such as a methanol synthesis catalyst comprising a solid support, or (ii) a bi-functional catalyst comprising, as a dehydration-functional constituent, a zeolite or non-zeolitic molecular sieve, such metals other than Cu, Zn, Al, Pt, Pd, and/or Cr, and present in the amounts described above, may be, more particularly, (a) metal(s) other than Cu, Zn, Al, Pt, Pd, Cr, and Si; metal(s) other than Cu, Zn, Al, Pt, Pd, Cr, Si, Ti, Zr, Mg, Ca, and Sr; or metal(s) other than Cu, Zn, Al, Pt, Pd, Cr, Si, Ti, Zr, Mg, Ca, Sr, and Y, (b) metal(s) other than Cu, Zn, Al, Pt, Pd, Cr, Si, and P; metal(s) other than Cu, Zn, Al, Pt, Pd, Cr, Si, P, Mg, Zn, Fe, Co, Ni, and Mn; or metal(s) other than Cu, Zn, Al, Pt, Pd, Cr, Si, P, Mg, Zn, Fe, Co, Ni, Mn, and Y, or (c) metal(s) other than Cu, Zn, Al, Pt, Pd, Cr, and Y; metal(s) other than Cu, Zn, Al, Pt, Pd, Cr, Mn, Mg, and Si; or metal(s) other than Cu, Zn, Al, Pt, Pd, Cr, Y, Mn, Mg, and Si. For convenience, in these particular embodiments, Si will be considered a “metal” in terms of its contribution to an alcohol synthesis catalyst, such as a methanol synthesis catalyst, or bi-functional catalyst.

A representative dehydration catalyst or dehydration-functional constituent of a bi-functional catalyst may comprise a zeolite (zeolitic molecular sieve) or a non-zeolitic molecular sieve (zeotype). Particular zeolites or non-zeolitic molecular sieves may have a structure type selected from the group consisting of CHA, TON, FAU, FER, BEA, EM, MFI, MEL, MTW, MWW, MOR, LTL, LTA, EMT, MAZ, MEI, AFI, and AEI, and preferably selected from one or more of CHA, TON, FAU, FER, BEA, EM, MEI, MOR, and MEI. The structures of zeolites having these and other structure types are described, and further references are provided, in Meier, W. M, et al., Atlas of Zeolite Structure Types, 4^{th} Ed., Elsevier: Boston (1996). Specific examples include SSZ-13 (CHA structure), zeolite Y(FAU structure), zeolite X(FAU structure), MCM-22 (MWW structure), zeolite beta (BEA structure), ZSM-5 (MFI structure), and ZSM-22 (TON structure), with zeolite beta and ZSM-5 being exemplary.

Non-zeolitic molecular sieves (zeotypes) include ELAPO molecular sieves which are embraced by an empirical chemical composition, on an anhydrous basis, expressed by the formula:

(EL_{X}Al_{y}P_{z})O₂

wherein EL is an element selected from the group consisting of silicon, magnesium, zinc, iron, cobalt, nickel, manganese, chromium and mixtures thereof, x is the mole fraction of EL and is often at least 0.005, y is the mole fraction of aluminum and is at least 0.01, z is the mole fraction of phosphorous and is at least 0.01 and x+y+z=1. When EL is a mixture of metals, x represents the total mole fraction of such metals present. The preparation of various ELAPO molecular sieves is known, and examples of synthesis procedures and their end products may be found in U.S. Pat. No. 5,191,141 (ELAPO); U.S. Pat. No. 4,554,143 (FeAPO); U.S. Pat. No. 4,440,871 (SAPO); U.S. Pat. No. 4,853,197 (MAPO, MnAPO, ZnAPO, CoAPO); U.S. Pat. No. 4,793,984 (CAPD); U.S. Pat. Nos. 4,752,651 and 4,310,440. Preferred ELAPO molecular sieves are SAPO and ALPO molecular sieves. Generally, the ELAPO molecular sieves are synthesized by hydrothermal crystallization from a reaction mixture containing reactive sources of EL, aluminum, phosphorus and a templating agent. Reactive sources of EL are the metal salts of EL elements defined above, such as their chloride or nitrate salts. When EL is silicon, a preferred source is fumed, colloidal or precipitated silica. Preferred reactive sources of aluminum and phosphorus are pseudo-boehmite alumina and phosphoric acid. Preferred templating agents are amines and quaternary ammonium compounds. An especially preferred templating agent is tetraethylammonium hydroxide (TEAOH).

A particularly preferred dehydration catalyst or dehydration-functional constituent comprises an ELAPO molecular sieve in which EL is silicon, with such molecular sieve being referred to in the art as a SAPO (silicoaluminophosphate) molecular sieve. In addition to those described in U.S. Pat. Nos. 4,440,871 and 5,191,141, noted above, other SAPO molecular sieves that may be used are described in U.S. Pat. No. 5,126,308. Of the specific crystallographic structures described in U.S. Pat. No. 4,440,871, SAPO-34, i.e., structure type 34, represents a preferred component of an LPG synthesis catalyst system. The SAPO-34 structure (CHA structure) is characterized in that it adsorbs xenon but does not adsorb iso-butane, indicating that it has a pore opening of about 4.2 Å. Accordingly, a representative dehydration catalyst or dehydration-functional constituent of a bi-functional catalyst may comprise SAPO-34 or other SAPO molecular sieve, such as SAPO-17, which is likewise disclosed in U.S. Pat. No. 4,440,871 and has a structure characterized in that it adsorbs oxygen, hexane, and water but does not adsorb iso-butane, indicative of a pore opening of greater than about 4.3 Å and less than about 5.0 Å. Due to its acidity, SAPO-34 can catalyze the conversion of an alcohol intermediate, such as a methanol intermediate, to olefins such as propylene. Without being bound by theory, it is believed that the characteristic hydrogen partial pressures used in the LPG synthesis stage not only promote the hydrogenation of these olefins, but also stabilize the dehydration catalyst/functional constituent by preventing coking. According to particular embodiments, the dehydration catalyst or dehydration-functional constituent may comprise a zeolite (zeolitic molecular sieve) of ZSM-5 or SSZ-13 or a non-zeolitic molecular sieve (zeotype) of SAPO-34 or SAPO-17. With respect to any particular zeolite or non-zeolitic molecular sieve that is used in an LPG synthesis catalyst system described herein, this may be present in any form according to which ion exchange sites are in their hydrogen form or otherwise exchanged with a suitable cation, non-limiting examples of which are cations of alkali metals (e.g., Na t), cations of alkaline earth metals (e.g., Ca′), and ammonium cation (NH₄⁺). For example, as a zeolite, hydrogen form SSZ-13 (HSSZ-13) may be used; as a non-zeolitic molecular sieve, hydrogen form SAPO-34 (HSAPO-34) may be used.

According to preferred embodiments, in the case of the dehydration catalyst or dehydration-functional constituent comprising a zeolite or a non-zeolitic molecular sieve, a stabilizer may be present in ion-exchange sites thereof, i.e., the dehydration catalyst or dehydration-functional constituent may comprise an ion-exchanged zeolite or an ion-exchanged non-zeolitic molecular sieve, having been prepared by ion-exchange to achieve a desired distribution of the stabilizer(s), such as a particularly preferred distribution of a noble metal (e.g., platinum), within the zeolite or non-zeolitic molecular sieve. The technique of ion-exchange can be used to advantageously influence the efficiency and single-atom properties of dispersed metal that is incorporated throughout a zeolite or non-zeolitic molecular sieve support material, leading to performance advantages. In this regard, a single metal atom or ion, or even a larger metal nanoparticle, will have a different electronic structure compared to a cluster of metal atoms, and consequently the catalytic behavior will likewise differ. The catalytic activity of an isolated, single metal atom or ion will depend on its coordination environment which, in turn, is governed by its location within the support material. Other preparation techniques for metal loading, such as incipient wetness impregnation and co-precipitation, involve dissolving precursor salts of the metal in a solvent, followed by precipitating the metal or metal salts onto the support material by a mechanism such as evaporation of the solvent or causing a reaction to decrease solubility of the metal. These techniques typically result, for a given sample, in a distribution of metal nanoparticle sizes across a corresponding distribution in the support environment. The amount of metal loaded or deposited is governed by the quantity of precursor salt that is dissolved and contacted with a given amount of the support material.

With respect to ion-exchange as a preparation technique, zeolites and non-zeolitic molecular sieves have well-defined structures, including pore geometries, with the presence of heteroatoms in their silica networks, most notably Al, that cause a charge imbalance. This can be compensated for by having additional cations, other than Al′, within micropores of the support material. In the case of protons (H f), these cations result in acidity. More generally, however, ion-exchange can be used to incorporate any of a number of possible cations within a zeolite or non-zeolitic molecular sieve. According to this technique, such support material is immersed in a solution of a cation, different from that already present in the exchange sites of the support material, and cations of the exchange sites are replaced with (exchanged by) cations of the solution, to equilibrium. The zeolite or non-zeolitic molecular sieve can then be washed to remove all ionic species that are not electrostatically bound to heteroatom exchange sites within the pores of the support material. Using ion-exchange, stabilizers described herein (e.g., platinum) and catalytically active metals generally may be effectively loaded onto a zeolite or non-zeolitic molecular sieve support material. An important distinction between ion-exchange and other catalyst preparation techniques, such as incipient wetness impregnation and co-precipitation, is the ability of ion-exchange to deposit active metals as single (atomic) cations and at specific sites on the catalyst, namely those sites with a charge imbalance resulting from a heteroatom such as Al. The deposited metal is therefore atomically disperse and present in a limited number of specific coordination environments. With other catalyst preparation techniques, no charge balancing is required, and therefore the use of precursor salts can result in metals clumping together, at indiscriminate locations of the support material, causing the formation of clusters of metal atoms, or possibly metal compounds (e.g., metal oxides) following activation. Therefore, a zeolite or non-zeolitic molecular sieve, which has been ion-exchanged with a stabilizer or other metal may be characterized by a high dispersion of the loaded species at sites having particular electronic characteristics. The structural differences, in terms of this metal dispersion resulting from ion-exchange versus other techniques for preparing a metal-loaded zeolite or metal-loaded non-zeolitic molecular sieve, can impart corresponding differences in terms of performance of the resulting catalyst. Such performance differences may manifest, for example, as improved selectivity, productivity, and/or yield of LPG hydrocarbons, resulting from the use of an ion-exchanged zeolite or non-zeolitic molecular sieve. For example, non-selective reactions such as methane formation may be desirably suppressed.

Further in contrast to other catalyst preparation techniques, ion-exchange imparts certain limits, based on the available ion-exchange sites, with respect to the amount of metal that can be deposited by ion-exchange on a given zeolite or non-zeolitic molecular sieve. The concentration of these exchange sites, for example in the case of a zeolite, may be directly correlated to its silica to alumina (SiO₂/Al₂O₃) molar framework ratio, with the presence of the Al heteroatoms giving rise to exchange sites as described above. More particularly, whereas each SiO₂ unit of the support material is neutral, each AlO₂ unit produces a negative charge, to be balanced by cations to be exchanged (e.g., Pt cations), or optionally cationic groups to be exchanged, such as cation-ligand groups (e.g., cation-nitrate groups including Pt-nitrate groups), cation-oxygen groups (e.g., Pt-oxygen groups), or other groups in which a cation such as platinum in any of its normal valence states (e.g., +2, +3, or +4) is incorporated within the micropores of the support material. In general, valences of such cationic groups as a whole, for example Pt-nitrate groups, may be minimally+1, such that ion-exchange is practically limited stoichiometrically to as many as one metal atom per exchange site. In the case of a dehydration catalyst or dehydration-functional constituent comprising a zeolite or non-zeolitic molecular sieve, the use of an ion-exchange preparation technique, causing the stabilizer to be present in ion-exchange sites, results in the structural distinctions described above, which are namely characteristic of an ion-exchanged zeolite or ion-exchanged non-zeolitic molecular sieve. The overall structure resulting from ion-exchange, including pore volume, is largely the same as that of the support material prior to ion-exchange, with the main difference being the size and type of cation being present within the micropores. In preferred embodiments, a zeolite component of a dehydration catalyst or dehydration-functional constituent is zeolite beta, and/or a stabilizer that is present in ion-exchange sites of zeolite or non-zeolitic molecular sieve is platinum.

In the case of the dehydration catalyst or dehydration-functional constituent comprising a zeolite or a non-zeolitic molecular sieve, such catalyst or functional constituent may be more particularly defined as a solid acid dehydration catalyst or solid acid dehydration-functional constituent, on the basis of the acidity exhibited by the zeolite or non-zeolitic molecular sieve (e.g., prior to ion-exchange). The acidity of a given zeolite or non-zeolitic molecular sieve may be determined, for example, by temperature programmed desorption (TPD) of a quantity of ammonia (ammonia TPD), from an ammonia-saturated sample of the material, over a temperature from 275° C. (527° F.) to 500° C. (932° F.), which is beyond the temperature at which the ammonia is physisorbed. The quantity of acid sites, in units of micromoles of acid sites per gram (μmol/g) of material, therefore corresponds to the number of micromoles of ammonia that is desorbed per gram of material in this temperature range. Alternatively, acidity may be calculated from, or based on, framework cation concentration of the zeolite or non-zeolitic molecular sieve. For example, in the particular case of the zeolite silicalite having a silica to alumina (SiO₂/Al₂O₃) molar framework ratio of 2000:1 (i.e., an Si/Al molar ratio of 1000:1), this would correspond to 16.6 μmol/g of acid sites, on the basis of the concentration of Al⁺³ cations. According to the TPD analysis above, in the absence of oligomerization, one NH₃ molecule would theoretically be absorbed per acid site or Al⁺³ cation in the above example. A representative zeolitic or non-zeolitic molecular sieve, or otherwise a representative dehydration catalyst or dehydration-functional constituent, has at least about 15 μmol/g (e.g., from about 15 to about 75 μmol/g) of acid sites, or at least about 25 μmol/g (e.g., from about 25 to about 65 μmol/g) of acid sites, measured by ammonia TPD or otherwise based on framework cation concentration. As noted above, in the case of zeolitic molecular sieves, acidity is a function of the silica to alumina (SiO₂/Al₂O₃) molar framework ratio, and, in embodiments in which the dehydration catalyst or dehydration-functional constituent comprises a zeolitic molecular sieve, its silica to alumina molar framework ratio may be less than about 2400 (e.g., from about 1 to about 2400), less than about 1000 (e.g., from about 1 to about 1000), less than about 400 (e.g., from about 1 to about 400), less than about 60 (e.g., from about 1 to about 60), or less than about (e.g., from about 5 to about 40).

According to preferred embodiments, a dehydration catalyst (ATLPG catalyst, such as an MTLPG catalyst) or a dehydration-functional constituent (ATLPG-functional constituent, such as an MTLPG-functional constituent) may comprise one or more stabilizers such as a noble metal stabilizer (e.g., platinum) or a non-noble metal stabilizer (e.g., yttrium in elemental form, in the form of an oxide such as yttria or carbonate or other form) on a support (e.g., a solid acid support) comprising a zeolite or non-zeolitic molecular sieve. For example, the stabilizer(s) (e.g., platinum and/or yttrium) in elemental form or in a compound form may be dispersed uniformly or non-uniformly on such support. The stabilizer(s) (e.g., platinum and/or yttrium) in these embodiments may be present in such ATLPG catalyst (e.g., MTLPG catalyst) or ATLPG-functional constituent (e.g., MTLPG-functional constituent) in an amount, or a combined amount, as described herein, such as from about 0.03 wt-% to about 15 wt-%, from about 0.1 wt-% to about 10 wt-%, from about 0.5 wt-% to about 5 wt-%, or from about 1 wt-% to about 3 wt-%, based on the weight of the stabilizer(s), relative to the weight of the catalyst or functional constituent. Such amounts of stabilizer(s) may be representative of amounts deposited or incorporated into the zeolite or non-zeolitic molecular sieve by any catalyst preparation technique, such as incipient wetness impregnation, co-precipitation, or ion-exchange. A preferred technique is ion-exchange that results in certain structural distinctions described above, in terms of the distribution of the stabilizer(s) at available ion-exchange sites. In the case of ion-exchange, the amounts of one or more stabilizers may be characterized in terms of the ion-exchange capacity of the zeolite or non-zeolitic molecular sieve, as would be appreciated by those skilled in the art, having knowledge of the present disclosure. In this regard, the one or more stabilizer(s) may be present in a dehydration catalyst (ATLPG catalyst, such as an MTLPG catalyst) or a dehydration-functional constituent (ATLPG-functional constituent, such as an MTLPG-functional constituent) in an amount representing at least about 70%, at least about 80%, at least about 90%, or at least about 95%, of the ion-exchange capacity of the zeolite or non-zeolitic molecular sieve as a component of such catalyst or functional constituent. Optionally, such catalyst or functional constituent may further comprise one or more promoters selected from the group consisting of manganese (Mn), magnesium (Mg), and silicon (Si), said promoter(s) being independently in elemental form or a compound form (e.g., oxide form).

Other than zeolitic and/or non-zeolitic molecular sieves, representative dehydration catalysts or dehydration-functional constituents may comprise one or more metal oxides, for example those selected from the group consisting of aluminum oxide, silicon oxide, titanium oxide, zirconium oxide, magnesium oxide, calcium oxide, iron oxide, vanadium oxide, chromium oxide, nickel oxide, tungsten oxide, and strontium oxide. Such metal oxides may serve as a binder to provide a structured dehydration catalyst or dehydration-functional constituent, and these metal oxides may, more particularly, serve as a binder for the zeolitic and/or non-zeolitic molecular sieve, if used to form the dehydration catalyst or dehydration-functional constituent. In representative embodiments, the dehydration catalyst or dehydration-functional constituent may comprise (a) a single type of zeolitic molecular sieve or (b) a single type of non-zeolitic molecular sieve, with (a) or (b) optionally being in combination with (c) a single type of metal oxide. In this case, (a) or (b), and optionally (c), may be present in an amount, or optionally a combined amount, of greater than about 75 wt-% (e.g., from about 75 wt-% to about 99.9 wt-%) or greater than about 90 wt-% (e.g., from about 90 wt-% to about 99 wt-%), based on the weight of the dehydration catalyst or dehydration-functional constituent. For example, according to more particular embodiments, (a) or (b) alone may be present in these representative amounts.

The dehydration catalyst or a dehydration-functional constituent may further comprise one or more stabilizers (e.g., in elemental form such as elemental platinum, or in the form of an oxide such as yttria or other form) and/or one or more promoters selected from the group consisting of Mn, Mg, and/or Si (e.g., independently in elemental forms, oxide forms, or other forms), in respective amounts as described above. In general, in the case of a dehydration catalyst or dehydration-functional constituent of a bi-functional catalyst, the zeolitic and/or non-zeolitic molecular sieve(s), together with optionally one or more metal oxides as described above, stabilizers such as platinum or yttrium (e.g., in elemental form such as elemental platinum, or in the form of an oxide such as yttria or other form), and/or one or more promoters selected from the group consisting of manganese (Mn), magnesium (Mg), and silicon (Si) (e.g., independently in elemental forms, oxide forms, or other forms), may be present in a combined amount representing at least about 90%, at least about 95%, or at least about 99%, of the total weight of the dehydration catalyst or dehydration-functional constituent.

In the case of the dehydration catalyst or dehydration-functional constituent comprising a zeolite or a non-zeolitic molecular sieve, the zeolite or non-zeolitic molecular sieve may provide a solid support for components of this catalyst or functional constituent, such as one or more stabilizers and/or one or more promoters as described herein. For example, such stabilizer(s) and/or promoter(s) may be incorporated in the zeolite or non-zeolitic molecular sieve, acting as a solid support, according to known techniques for catalyst preparation, including sublimation, impregnation, or dry mixing. In the case of impregnation, which is an exemplary technique, an impregnation solution of soluble compounds of the one or more stabilizers and/or one or more promoters in a polar (aqueous) or non-polar (e.g., organic) solvent may be contacted with the solid support, preferably under an inert atmosphere. For example, this contacting may be carried out, preferably with stirring, in a surrounding atmosphere of nitrogen, argon, and/or helium, or otherwise in a non-inert atmosphere, such as air. The solvent may then be evaporated from the solid support, for example using heating, flowing gas, and/or vacuum conditions, leaving the dried, solid support comprising the zeolite or non-zeolitic molecular sieve and being impregnated with the stabilizer(s) and/or promoter(s). These components may be impregnated in the solid support, such as in the case of a plurality of metals (e.g., one or more stabilizers and one or more promoters, or otherwise two or more stabilizers or two or more promoters) being impregnated simultaneously by being dissolved in the same impregnation solution, or otherwise being impregnated separately using different impregnation solutions and contacting steps. In any event, the zeolite or non-zeolitic molecular sieve, acting as the solid support for impregnated stabilizer(s) and/or promoter(s) may be subjected to further preparation steps, such as washing with the solvent to remove excess metal(s) and impurities, further drying, calcination, etc. to provide the dehydration catalyst or dehydration-functional constituent.

In yet further embodiments, as an alternative to supporting stabilizer(s) and/or promoter(s), or in addition to supporting stabilizer(s) and/or promoter(s), the zeolite or non-zeolitic molecular sieve may support one or more transition metals (e.g., one or more of Pt, Pd, Rh, Ir, and/or Au) in elemental form or in a compound form. Such one or more transition metals may be present in an amount, or combined amount, from about 0.05 wt-% to about 5 wt-%, such as from about 0.1 wt-% to about 3 wt-%, based on the weight of the transition metal(s), relative to the weight of the dehydration catalyst or dehydration-functional constituent comprising the zeolite or non-zeolitic molecular sieve. In still further embodiments, as an alternative to supporting stabilizer(s), promoter(s), and/or transition metal(s) or in addition to supporting stabilizer(s), promoter(s), and/or transition metal(s), the zeolite or non-zeolitic molecular sieve may support one or more surface-modifying agents (e.g., one or more of Si, Na, and/or Mg) in elemental form or in a compound form. Any surface-modifying agents, which by definition are disposed predominantly or completely on an external surface of the zeolite or non-zeolitic molecular sieve, may be present in an amount, or combined amount, from about 0.05 wt-% to about 5 wt-%, such as from about 0.1 wt-% to about 3 wt-%, based on the weight of the surface-modifying agent(s), relative to the weight of the dehydration catalyst or dehydration-functional constituent comprising the zeolite or non-zeolitic molecular sieve. In contrast to surface-modifying agents, any of the stabilizer(s), promoter(s), and/or transition metal(s) may be disposed uniformly throughout the zeolite or non-zeolitic molecular sieve used as a component of a dehydration catalyst or dehydration-functional constituent, or may be disposed according to any other profile (e.g., radial concentration profile), such as predominantly or completely on an external surface of the zeolite or non-zeolitic molecular sieve, or otherwise predominantly or completely within internal pores of such zeolite or non-zeolitic molecular sieve.

Those skilled in the art having knowledge of the present disclosure, including the general catalyst preparation procedures described above, will appreciate how such procedures can be adapted to obtain loadings of components (e.g., stabilizer(s), promoter(s), and/or transition metal(s)) with a desired profile, such as in the case of being concentrated near the external surface of, concentrated within internal pores of, or disposed uniformly throughout, a zeolite or non-zeolitic molecular sieve, with the desired profile likewise being applicable to the dehydration catalyst or dehydration-functional constituent as a whole. For example, an impregnation solution may be contacted with a powder form, or other finely divided form, of the zeolite or non-zeolitic molecular sieve to obtain a uniform distribution. Otherwise, in the case of using any one or more of the metal oxides described above as a binder for the zeolite or non-zeolitic molecular sieve, an impregnation solution may be contacted with larger, structured forms of the bound zeolite or non-zeolitic molecular sieve (e.g., having dimensions equivalent to, or on the same order as, the dehydration catalyst or dehydration-functional constituent as a whole) to obtain distributions of components preferentially near the external surface of the zeolite or non-zeolitic molecular sieve, or otherwise the dehydration catalyst or dehydration-functional constituent.

In a representative dehydration catalyst or bi-functional catalyst, any metal(s) other than (a) Pt and/or Y (optionally together with any one or more other metals selected from Group 3 or Group 4 of the Periodic Table, and/or one or more lanthanides) and/or (b) metal(s) present in the zeolitic and/or non-zeolitic molecular sieve(s) and optionally one or more metal oxides as described above, may be present in minor amounts, may be substantially absent, or may be absent. For example, any such metal(s) other than (a) and/or (b) may be independently present in an amount of less than about 1 wt-%, less than about 0.1 wt-%, or even less than about 0.05 wt-%, based on the total catalyst weight. Alternatively, any two or more of such other metals may be present in a combined amount of less than about 2 wt-%, less than about 0.5 wt-%, or even less than about 0.1 wt-%, based on the total catalyst weight. According to particular embodiments, a dehydration catalyst or bi-functional catalyst may comprise metal(s) other than Pt and/or Y (optionally together with any one or more other metals selected from Group 3 or Group 4 of the Periodic Table, and/or one or more lanthanides), Mn, and/or Mg in the amounts described above; metals other than Pt and/or Y (optionally together with any one or more other metals selected from Group 3 or Group 4 of the Periodic Table, and/or one or more lanthanides), Mn, Mg, and/or Si in the amounts described above; metals other than Pt and/or Y (optionally together with any one or more other metals selected from Group 3 or Group 4 of the Periodic Table, and/or one or more lanthanides), Mn, Mg, Si, and/or P in the amounts described above; metals other than Pt and/or Y (optionally together with any one or more other metals selected from Group 3 or Group 4 of the Periodic Table, and/or one or more lanthanides), Mn, Mg, Si, P, Zn, Co, and/or Fe in the amounts described above; metals other than Pt and/or Y (optionally together with any one or more other metals selected from Group 3 or Group 4 of the Periodic Table, and/or one or more lanthanides), Mn, Mg, Si, P, Zn, Co, Fe, Al, Ti, Zr, Mg, and/or Ca in the amounts described above; or metals other than Pt and/or Y (optionally together with any one or more other metals selected from Group 3 or Group 4 of the Periodic Table, and/or one or more lanthanides), Mn, Mg, Si, P, Zn, Co, Fe, Al, Ti, Zr, Mg, Ca, V, Cr, Ni, W, and/or Sr in the amounts described above. For convenience, in these particular embodiments, Si and P will be considered “metals” in terms of their contributions to a dehydration catalyst or bi-functional catalyst. Other components of an alcohol synthesis catalyst (e.g., a methanol synthesis catalyst), a dehydration catalyst, or a bi-functional catalyst as described herein, such as binders (e.g., one or more metal oxides as described herein) and other additives, may be present in minor amounts, such as in an amount, or combined amount, of less than about 10 wt-% (e.g., from about 0.01 wt-% to about 10 wt-%), less than about 5 wt-% (e.g., from about 0.01 wt-% to about 10 wt-%), or less than about 1 wt-% (e.g., from about 0.01 wt-% to about 10 wt-%), based on the weight of the catalyst.

In the case of LPG synthesis catalyst systems comprising separate compositions of two different catalyst types, components of an alcohol synthesis catalyst (e.g., a methanol synthesis catalyst) as described herein may be substantially absent, or absent, from a dehydration catalyst. In the same manner, components of a dehydration catalyst as described herein may be substantially absent, or absent, from an alcohol synthesis catalyst (e.g., a methanol synthesis catalyst). For example, a representative dehydration catalyst may comprise (a) one or more alcohol synthesis-active metals (e.g., methanol synthesis-active metals(s)) described herein, (b) a solid support as described herein, (c) one or more stabilizers such as platinum and/or yttrium, and/or (d) one or more promoters of Mn, Mg, and/or Si, in an amount of (a), (b), (c), and/or (d), such as in a combined amount of (a), (b), (c), and (d), of less than about 5 wt-%, less than about 1 wt-%, or less than about 0.1 wt-%. This applies to dehydration catalysts generally, but this may also apply, more particularly, to dehydration catalysts of catalyst systems in which the alcohol synthesis catalyst (e.g., methanol synthesis catalyst) comprises, respectively, (a), (b), (c), and/or (d). For example, in the case of an alcohol synthesis catalyst (e.g., methanol synthesis catalyst) comprising (a) one or more alcohol synthesis-active metal(s) (e.g., methanol synthesis-active metals(s)), a dehydration catalyst of a catalyst system comprising that alcohol synthesis catalyst (e.g., methanol synthesis catalyst) may comprise such (a) one or more alcohol synthesis-active metal(s) (e.g., methanol synthesis-active metals(s)) in an amount, or combined amount, as described above (e.g., in an amount, or combined amount, of less than about 0.1 wt-%). Similarly, in the case of an alcohol synthesis catalyst (e.g., methanol synthesis catalyst) comprising (b), (c), and/or (d), a dehydration catalyst of a catalyst system comprising that alcohol synthesis catalyst (e.g., methanol synthesis catalyst) may comprise such respective (b), (c), and/or (d) in an amount, or combined amount, as described above. Alternatively, or in combination, a representative alcohol synthesis catalyst (e.g., methanol synthesis catalyst) may comprise (a) one or more zeolitic and/or non-zeolitic molecular sieve(s), (b) one or more metal oxides as described above, (c) one or more stabilizers such as platinum and/or yttrium, (d) one or more promoters of Mn, Mg, and/or Si, (e) one or more transition metals (e.g., Pt, Pd, Rh, Ir, and/or Au), and/or (f) one or more surface-modifying agents (e.g., Si, Na, and/or Mg) in an amount of (a), (b), (c), (d), (e), and/or (f) such as in a combined amount of (a), (b), (c), (d), (e), and/or (f) of less than about 5 wt-%, less than about 1 wt-%, or less than about 0.1 wt-%. This applies to alcohol synthesis catalysts (e.g., methanol synthesis catalysts) generally, but this may also apply, more particularly, to alcohol synthesis catalysts (e.g., methanol synthesis catalysts) of catalyst systems in which the dehydration catalyst comprises, respectively, (a), (b), (c), (d), (e), and/or (f). For example, in the case of a dehydration catalyst comprising (a) one or more zeolitic and/or non-zeolitic molecular sieve(s), an alcohol synthesis catalyst (e.g., methanol synthesis catalyst) of a catalyst system comprising that dehydration catalyst may comprise such (a) one or more zeolitic and/or non-zeolitic molecular sieve(s) in an amount, or combined amount, as described above (e.g., in an amount, or combined amount, of less than about 0.1 wt-%). Similarly, in the case of a dehydration catalyst comprising (b), (c), (d), (e), and/or (f), a methanol synthesis catalyst of a catalyst system comprising that dehydration catalyst may comprise such respective (b), (c), (d), (e), and/or (f) in an amount, or combined amount, as described above.

As an alternative to separate compositions of two different catalyst types, LPG synthesis may be performed using a single catalyst composition, namely a bi-functional catalyst comprising both an alcohol synthesis-functional constituent (e.g., methanol synthesis-functional constituent) and a dehydration-functional constituent. In terms of the compositions of these constituents, they may correspond in isolation to an alcohol synthesis catalyst (e.g., a methanol synthesis catalyst) and a dehydration catalyst, respectively, as described herein. When combined in a single catalyst composition, the functional constituents (i) and (ii) may be present in weight ratios as described herein. A representative bi-functional catalyst may therefore comprise (i) an alcohol synthesis-functional constituent (e.g., methanol synthesis-functional constituent) comprising one or more alcohol synthesis-active metals (e.g., methanol synthesis-active metals) as described above, and optionally a solid support as described herein, and (ii) a dehydration-functional constituent comprising a zeolite or non-zeolitic molecular sieve, and optionally a metal oxide, one or more transition metals, and/or one or more surface-modifying agents, as described herein. Either (i) or (ii) may further comprise a stabilizer such as platinum and/or yttrium (e.g., in elemental form such as elemental platinum, or in the form of an oxide such as yttria or other form) and/or one or more promoters selected from the group consisting of Mn, Mg, and/or Si (e.g., independently in elemental forms, oxide forms, or other forms).

It can be appreciated from the above description, including the weight ratios in which (i) and (ii) may be combined, that the one or more alcohol synthesis-active metals (e.g., methanol synthesis-active metals) may be present in a bi-functional catalyst as a whole, in an amount, or combined amounts, that is/are less than that/those amounts in which they are present in an alcohol synthesis catalyst (e.g., methanol synthesis catalyst), as described above. Likewise, the zeolite or non-zeolitic molecular sieve may be present in a bi-functional catalyst as a whole, in an amount that is less than that in which it is present in a dehydration catalyst, as described above. For example, a bi-functional catalyst as a whole may comprise the one or more alcohol synthesis-active metals (e.g., methanol synthesis-active metals) in lower amount, such as independently in an amount generally from about 0.2 wt-% to about 30 wt-%, typically from about 0.5 wt-% to about 15 wt-%, and often from about 0.8 wt-% to about 5 wt-%, based on the weight of the bi-functional catalyst. Likewise, a bi-functional catalyst as a whole may comprise (a) a single type of zeolitic molecular sieve or (b) a single type of non-zeolitic molecular sieve, with (a) or (b) optionally being in combination with (c) a single type of metal oxide. In this case, (a) or (b), and optionally (c), may be present in an amount, or optionally a combined amount, of greater than about 35 wt-% (e.g., from about 35 wt-% to about 95 wt-%), greater than about 50 wt-% (e.g., from about 50 wt-% to about 90 wt-%), or greater than about 75 wt-% (e.g., from about 75 wt-% to about 85 wt-%), based on the weight of the bi-functional catalyst. For example, according to more particular embodiments, (a) or (b) alone may be present in these representative amounts. Further in view of the above description, based on the weight of one or more stabilizers such as platinum and/or yttrium, and, relative to the weight of a bi-functional catalyst as a whole, the one or more stabilizers such as platinum and/or yttrium (e.g., in elemental form such as elemental platinum, or in the form of an oxide such as yttria or other form) may be present in an amount from about 0.01 wt-% to about 10 wt-%, such as from about 0.05 wt-% to about 6 wt-% or from about 0.1 wt-% to about 1 wt-%. Based on the weight of Mn, Mg, and/or Si, and, relative to the weight of the bi-functional catalyst as a whole, the promoter(s) (e.g., independently in elemental forms, oxide forms, or other forms) may be present in an amount from about 0.05 wt-% to about 12 wt-%, such as from about 0.1 wt-% to about 10 wt-% or from about 0.5 wt-% to about 8 wt-%.

Representative bi-functional catalysts may therefore comprise: (i) as an alcohol synthesis-functional constituent (e.g., methanol synthesis-functional constituent), one or more alcohol synthesis-active metals (e.g., methanol synthesis-active metal(s)), or any forms of such metals (e.g., elemental and/or oxide forms), as described herein, and optionally any solid support as described herein, and (ii) as a dehydration-functional constituent, one or more zeolitic and/or non-zeolitic molecular sieve(s) as described herein, and optionally one or more metal oxides, one or more transition metals, and/or one or more surface-modifying agents, as described herein. Such bi-functional catalyst may further comprise, for example as component(s) of either the alcohol synthesis-functional constituent (e.g., methanol synthesis-functional constituent) and/or the dehydration-functional constituent, a stabilizer such as platinum and/or yttrium (e.g., in elemental form such as elemental platinum, or in the form of an oxide such as yttria or other form), and/or one or more promoters of Mn, Mg, and/or Si (e.g., independently in elemental forms, oxide forms, or other forms). The one or more alcohol synthesis-active metals (e.g., methanol synthesis-active metal(s)), or any forms of such metals (e.g., elemental and/or oxide forms), one or more zeolitic and/or non-zeolitic molecular sieve(s), together with any optional solid support, optional metal oxide(s), optional transition metal(s), optional surface-modifying agent(s), stabilizers such as platinum and/or yttrium (e.g., in elemental form such as elemental platinum, in the form of an oxide such as yttria or other form), and/or one or more promoters of Mn, Mg, and/or Si (e.g., independently in elemental forms, oxide forms, or other forms), may constitute all or substantially all of the bi-functional catalyst, for example these components may be present in a combined amount representing at least about 90%, at least about 95%, or at least about 99%, of the total weight of the bi-functional catalyst.

Conditions used in processes for producing an LPG product, and more particularly conditions under which LPG synthesis catalyst systems, as described herein, are maintained (e.g., in one or more LPG synthesis reactors), are suitable for the conversion of H₂ and CO in a synthesis gas to propane and/or butane of the LPG product. In representative embodiments, such LPG synthesis reaction conditions, suitable for use in at least one LPG synthesis reactor or, more particularly, one or more catalyst beds contained in such reactor(s), can include an LPG synthesis reaction temperature in a range from about 204° C. (400° F.) to about 454° C. (850° F.), or from about 316° C. (600° F.) to about 399° C. (750° F.). These temperatures may be understood as referring to average (or weighted average) catalyst bed temperatures, and alternatively, according to some embodiments may be maximum or peak catalyst bed temperatures. An LPG synthesis reaction pressure, suitable for use in at least one LPG synthesis reactor, can include a gauge pressure from about 690 kPa (100 psig) to about 6.9 MPa (1000 psig), such as from about 1.38 MPa (200 psig) to about 2.76 MPa (400 psig) or from about 3.4 MPa (500 psig) to about 5.2 MPa (750 psig).

The LPG synthesis catalyst systems and LPG synthesis reaction conditions described herein are generally suitable for achieving a conversion of H₂ and/or CO (H₂ conversion or CO conversion) of at least about 20% (e.g., from about 20% to about 99% or from about 20% to about 95%), at least about 30% (e.g., from about 30% to about 99% or from about 30% to about 95%), or at least about 50% (e.g., from about 50% to about 95% or from about 75% to about 95%). As is understood in the art, the conversion of H₂ or CO in a synthesis gas can be calculated on the basis of:

100*(H_{2feed}−H_{2prod})/H_{2feed} or 100*(CO_{feed}−CO_{prod})/CO_{feed}

wherein H_{2feed} or CO_{feed} is the total amount (e.g., total weight or total moles) of H₂ or CO, respectively, in the synthesis gas provided to one or more LPG synthesis reactors containing an LPG synthesis catalyst system as described herein, and H_{2prod} or CO_{prod} is the total amount of H₂ or CO, respectively, in the effluent from the reactor(s), which may, but does not necessarily, correspond to the total amount of H₂ or CO in the LPG product. In the case of continuous processes, these total amounts may be more conveniently expressed in terms of flow rates, or total amounts per unit time (e.g., total weight/hr or total moles/hr). These H₂ or CO conversion levels may be based on “per-pass” conversion, achieved in a single pass through one or more LPG synthesis reactors, or otherwise based on overall conversion, achieved by returning a recycle portion of the effluent, containing unconverted H₂ and/or CO (and possibly enriched in these unconverted reactants, relative to the effluent and/or the LPG product), back to the LPG synthesis reactor(s). Whether these LPG synthesis conversion levels are based on H₂ conversion or CO conversion may depend on which reactant is stoichiometrically limited in the synthesis gas being fed or introduced to the LPG synthesis reactor(s), considering the LPG synthesis reaction chemistry. Preferably, these LPG synthesis conversion levels are based on CO conversion, or conversion of CO in the synthesis gas.

Another important performance parameter with respect to processes as described herein for producing an LPG product is carbon selectivity to LPG hydrocarbons, which refers to percentage of carbon (e.g., present in CO and CO₂) that is fed or introduced to the LPG synthesis reactor(s) and that manifests in LPG hydrocarbons, namely propane and/or butane (including both of the butane isomers, iso- and normal-butane) in the effluent from the reactor(s), which may, but does not necessarily, correspond to this percentage that manifests in LPG hydrocarbons in the LPG product. In representative embodiments, carbon selectivity to LPG hydrocarbons is at least about 20% (e.g., from about 20% to about 90% or from about 20% to about 75%), at least about 30% (e.g., from about 30% to about 90% or from about 30% to about 75%), at least about 40% (e.g., from about 40% to about 90% or from about 40% to about 75%), or even at least about 50% (e.g., from about 50% to about 90% or from about 50% to about 75%). The carbon selectivity to propane may be at least about 10% (e.g., from about 10% to about 60% or from about 10% to about 50%), at least about 15% (e.g., from about 15% to about 60% or from about 15% to about 50%), or at least about 20% (e.g., from about 20% to about 60% or from about 20% to about 50%). The carbon selectivity to butane (both iso- and normal-butane) may be at least about 5% (e.g., from about 5% to about 45% or from about 5% to about 35%), at least about 10% (e.g., from about 10% to about 45% or from about 10% to about 35%), or at least about 15% (e.g., from about 15% to about 45% or from about 15% to about 35%).

A per-pass (or single pass) yield of LPG hydrocarbons provides a further, important measure of performance of representative processes as described herein. This per-pass yield refers to the product of the per-pass CO conversion and the carbon selectivity to LPG hydrocarbons. In representative processes, the per-pass yield of LPG hydrocarbons (or LPG hydrocarbon yield) is at least about 15% (e.g., from about 15% to about 85% or from about 15% to about 70%), at least about 25% (e.g., from about 25% to about 85% or from about 25% to about 70%), at least about 35% (e.g., from about 35% to about 85% or from about 35% to about 70%), or even at least about 45% (e.g., from about 45% to about 85% or from about 45% to about 70%). In some preferred embodiments, the per-pass yield of LPG hydrocarbons in the LPG synthesis stage is at least about 50%.

A desired H₂ conversion and/or CO conversion in the LPG synthesis reactor(s), as well as other desired performance parameters, may be achieved by adjusting the LPG synthesis reaction conditions described above (e.g., LPG synthesis reaction temperature and/or LPG synthesis reaction pressure), and/or adjusting the weight hourly space velocity (WHSV). As is understood in the art, the WHSV is the weight flow of the synthesis gas divided by the total weight of catalyst in the LPG synthesis catalyst system (e.g., present in a fixed bed or other reactor bed configuration in the LPG synthesis reactor(s)) and represents the equivalent catalyst bed weights of the synthesis gas processed per hour. The WHSV is related to the inverse of the reactor residence time. The LPG synthesis reaction conditions may include a weight hourly space velocity (WHSV) generally less than about 10 hr⁻¹ (e.g., from about 0.01 hr⁻¹ to about 10 hr⁻¹), typically less than about 5 hr⁻¹ (e.g., from about 0.05 hr⁻¹ to about 5 hr⁻¹), and often less than about 1.5 hr⁻¹ (e.g., from about 0.1 hr⁻¹ to about 1.5 hr⁻¹), as defined above. As an alternative to being based on the entire weight of the catalyst in the LPG catalyst system, the WHSV may be based on the combined weight of a methanol synthesis catalyst and a dehydration catalyst, or otherwise based on the weight of a bi-functional catalyst, as described herein. The conversion level (e.g., CO conversion) may be increased, for example, by increasing pressure and decreasing WHSV, having the effects, respectively, of increasing reactant concentrations and reactor residence times.

The LPG product may be an LPG synthesis effluent, i.e., the effluent from one or more LPG synthesis reactors (e.g., the LPG product may be obtained without further processing of the LPG synthesis effluent) or otherwise the LPG product may be separated from the LPG synthesis effluent, for example as a fraction of the LPG synthesis effluent that is enriched in propane and/or butane and that is separated using techniques known in the art (e.g., fractionation). In either case, the LPG synthesis effluent may be obtained directly from an LPG synthesis reactor that contains an LPG synthesis catalyst system, or at least one catalyst of such system (e.g., an alcohol synthesis catalyst, such as a methanol synthesis catalyst, or a dehydration catalyst), as described herein. In preferred embodiments, processes described herein comprise a step of separating the LPG product from the LPG synthesis effluent. In addition to this LPG product, processes may further comprise separating one or more other fractions from the LPG synthesis effluent, such as fractions that are depleted in LPG hydrocarbons, relative to the LPG product. For example, such other fraction(s) may include an Hz/CO-enriched fraction, i.e., a fraction that is enriched in H₂ and CO, relative to the LPG synthesis effluent and the LPG product. Such other fraction(s) may, alternatively or in combination, include a water-enriched fraction, i.e., a fraction that is enriched in water, relative to the LPG synthesis effluent and the LPG product. Both such Hz/CO-enriched fraction and water-enriched fraction represent fractions that, following their separation from the LPG synthesis effluent, may advantageously be recycled in the process. The H₂/CO₂-enriched fraction and water-enriched fractions may, respectively, represent gaseous (vapor) and liquid fractions separated from the LPG synthesis effluent, e.g., as respective, lower-boiling (more volatile) and higher-boiling (less volatile) fractions, relative to the LPG product.

According to specific embodiments, the LPG product (e.g., following a step of separating the LPG product from the LPG synthesis effluent) may comprise propane and butane in a combined amount of at least about 60 mol-% (e.g., from about 60 mol-% to about 100 mol-%), at least about 80 mol-% (e.g., from about 80 mol-% to about 100 mol-%), or at least about 90 mol-% (e.g., from about 90 mol-% to about 99 mol-%). Together with such combined amounts, or alternatively, the LPG product may comprise propane and/or butane independently in individual amounts of at least about 25 mol-% (e.g., from about 25 mol-% to about 85 mol-%), at least about 40 mol-% (e.g., from about 40 mol-% to about 80 mol-%), or at least about 50 mol-% (e.g., from about 50 mol-% to about 75 mol-%). The balance of the LPG product may comprise all, or substantially all, pentane or a combination of ethane and pentane. According to other specific embodiments, at least about 40% (e.g., from about 40% to about 95%), at least about 55% (e.g., from about 55% to about 95%), or at least about 70% (e.g., from about 70% to about 95%) of the carbon content of the synthesis gas (e.g., the carbon content of CO and/or CO₂ present in this mixture) forms propane and/or butane of the LPG product. These percentages are equivalently expressed in terms of wt-% or mol-%.

### EXAMPLES

The following examples are set forth as representative of the present invention. These examples are not to be construed as limiting the scope of the invention as other equivalent embodiments will be apparent in view of the present disclosure and appended claims.

### LPG Synthesis Catalyst Systems and Performance Evaluation

An LPG synthesis catalyst system of 1 gram of methanol synthesis catalyst (Cu/ZnO/Al₂O₃), 3 grams of zeolite beta, and 1 gram of sand, contained in an LPG synthesis reactor, was tested for its activity to convert a 2:1 H₂:CO molar ratio synthesis gas. In separate tests of Examples 1-3, normal flow rates of the synthesis gas in ml/min of 165, 110, and 55 were used, respectively, in conjunction with other LPG synthesis conditions of 2.1 MPa (300 psig) gauge pressure and 350° C. (662° F.) catalyst bed temperature. Results are summarized in Table 1 below, including CO conversion and percent carbon selectivity for various components of the effluent obtained from the LPG synthesis reactor.

TABLE 1
Variations in synthesis gas flow rate

| Example | 1 | 2 | 3 |
|---|---|---|---|
| Flow rate, ml/min | 165 | 110 | 55 |
| WHSV, hr⁻¹ (based on 4 g catalyst) | 1.1 | 0.71 | 0.35 |
| CO Conversion | 83.5% | 88.0% | 91.4% |
| | Carbon Selectivity | | |
| CH₄ | 6% | 3% | 4% |
| CO₂ | 45% | 41% | 39% |
| ethane | 7% | 9% | 9% |
| propane | 22.4% | 27.9% | 29.8% |
| i-butane | 12.4% | 11.6% | 10.4% |
| n-butane | 5.5% | 6.7% | 7.0% |
| i-pentane | 1% | 1% | 1% |
| n-pentane | 0.0% | 0.0% | 0.0% |
| 2-methyl-pentane | 0.1% | 0.1% | 0.1% |
| 3-methyl-pentane | 0.0% | 0.1% | 0.0% |
| methanol | 0.0% | 0.1% | 0.0% |
| LPG hydrocarbons | 40% | 46% | 47% |
| C3 fraction of LPG | 0.56 | 0.60 | 0.63 |
| LPG Yield | 34% | 40% | 43% |

As is apparent from these results, the exemplary LPG synthesis catalyst system was active under the conditions described above, for converting synthesis gas to LPG hydrocarbons (propane and the iso- and normal-butane isomers) with a favorable CO conversion in a range of about 83-92% and carbon selectivity in a range of about 40-48%. Whereas it is believed that a methanol synthesis and dehydration reaction mechanism accounted for the production of these and other hydrocarbons, it is evident that the methanol intermediate was present in the effluent of the LPG synthesis reactor in only trace or undetectable quantities. In addition, these results illustrate the impact of reducing the rate of the synthesis gas fed or introduced to the LPG synthesis reactor. In particular, lowering the feed rate had the effect of increasing CO conversion, at least in part due to the increase in reactor residence time (decrease in WHSV). As would be understood by those skilled in the art having knowledge of the present disclosure, the feed rate and other LPG synthesis conditions can be varied to achieve other ranges of conversion levels.

The experiment described above in Example 1 and performed with a normal flow rate of the synthesis gas of 165 ml/min, was used as a baseline experiment for comparison purposes. Specifically, the 2:1 H₂:CO molar ratio synthesis gas, i.e., BASELINE FEED having an approximate H₂/CO composition of 67 mol-%/33 mol-%, was varied in subsequent experiments, in terms of its composition, to evaluate differences in performance that could be obtained. These feeds had compositions of:
(A) (i) 50 mol-% of 2:1 H₂:CO molar ratio synthesis gas, combined with (ii) 50 mol-% CO₂—FEED A, having an approximate H₂/CO₂/CO composition of 33.5 mol-%/50 mol-%/16.5 mol-% (Example 4);
(B) a 3:1 H₂:CO molar ratio synthesis gas—FEED B, having an approximate H₂/CO composition of 75 mol-%/25 mol-% (Example 5); and
(C) (i) 2:1 H₂:CO molar ratio synthesis gas, combined with (ii) an H₂/CO₂-enriched fraction of an effluent of the LPG synthesis reactor and representative of a synthesis gas obtained from recycle operation—FEED C, having an approximate H₂/CO₂/CO composition of 64 mol-%/20.5 mol-%/15.5 mol-% (Example 6).

Therefore, compared to BASELINE FEED, as can be appreciated from the above description, FEED A, FEED B, and FEED C were representative of comparative types of synthesis gas having, respectively, (i) an added amount of CO₂, (ii) an added amount of H₂, and (iii) added amounts of both H₂ and CO₂, as would be obtained from recycle of a fraction of the effluent of the LPG reactor, and particularly such fraction being enriched in H₂ and CO₂, relative to the effluent. These feeds were evaluated with respect to their conversion to LPG hydrocarbons and other components, under LPG synthesis conditions of 2.1 MPa (300 psi) gauge pressure and 350° C. (662° F.) catalyst bed temperature. These conditions were maintained in the presence of the exemplary LPG synthesis catalyst system of 1 gram of methanol synthesis catalyst (Cu/ZnO/Al₂O₃), 3 grams of zeolite beta, and 1 gram of sand, to carry out the LPG synthesis reaction. Results are summarized in Table 2 below, including CO conversion and percent carbon selectivity for various components of the effluent obtained from the LPG synthesis reactor.

TABLE 2
Variations in synthesis gas composition

| | Composition, | | | |
|---|---|---|---|---|
| | mol-% H₂/mol-% CO₂/mol-% CO | | | |
| | BASELINE, | FEED A, | FEED B, | FEED C, |
| | 67/0/33 | 33.5/50/16.5 | 75/0/25 | 64/20.5/15.5 |
| Example | 1 | 4 | 5 | 6 |
| Flow rate, | 165 | 165 | 165 | 165 |
| ml/min | | | | |
| CO Conversion | 83.5% | 19.7% | 84.3% | 51.6% |
| | Carbon Selectivity | | | |
| CH₄ | 6% | 25% | 7% | 13% |
| CO₂ | 45% | N/A | 42% | 6% |
| ethane | 7% | 17% | 6% | 12% |
| propane | 22.4% | 32.6% | 25.0% | 40.0% |
| i-butane | 12.4% | 18.3% | 14.3% | 21.1% |
| n-butane | 5.5% | 6.5% | 5.1% | 6.9% |
| i-pentane | 1% | 1% | 1% | 1% |
| n-pentane | 0.0% | 0.0% | 0.5% | 0.0% |
| 2-methyl-pentane | 0.1% | 0.1% | 0.1% | 0.2% |
| 3-methyl-pentane | 0.0% | 0.1% | 0.0% | 0.1% |
| methanol | 0.0% | 0.0% | 0.0% | 0.0% |
| LPG | 40% | 57% | 44% | 68% |
| hydrocarbons | | | | |
| C3 fraction | 0.56 | 0.57 | 0.56 | 0.59 |
| of LPG | | | | |
| LPG Yield | 34% | 11% | 37% | 35% |

From the above results, it is evident that, compared to the BASELINE FEED, adding CO₂ alone to obtain FEED A (Example 4) caused a significant reduction in the rate of the LPG synthesis reaction and therefore the CO conversion. It is believed that this effect was due not only to the dilution of the CO reactant and corresponding decrease in its concentration or partial pressure in the reaction mixture, but also to a suppression by CO₂ of the LPG synthesis reaction. Therefore, in cases of compositions of synthesis gas having a significant contribution of CO₂, a substantial increase in catalyst, or otherwise a substantial decrease in feed rate (throughput), could be required to establish baseline CO conversion levels obtained with purely H₂- and CO-containing synthesis gas alone. At the lower CO conversion levels observed with FEED A compared to the BASELINE FEED, some increase in selectivity to LPG hydrocarbons was observed, although significantly greater amounts of methane and ethane were also produced. With respect to the addition of H₂ alone to the BASELINE FEED, according to the results obtained with FEED B (Example 5), use of the 3:1 H₂:CO molar ratio synthesis gas did not cause a reduction in reaction rate, as CO conversion was comparable to that obtained with the BASELINE FEED. Nor did the addition of H₂ alone reduce the formation of CO₂, via the water-gas shift reaction. To the extent that increased H₂ concentration might drive this reaction toward CO and H₂O production, the additional H₂ also effectively displaced some CO, with the overall effect being that this additional H₂ acted essentially as an inert gas.

Surprisingly, however, compared to the BASELINE FEED, adding CO₂ and H₂ in combination to obtain FEED C (Example 6), despite causing a CO conversion deficit, resulted in nearly 70% selectivity to LPG hydrocarbons, with little or no generation of CO₂ through the LPG synthesis reactor. Whereas the selectivity to all C₁-C₄ hydrocarbons increased, the ratio of CH₄ and ethane to LPG hydrocarbons was essentially unchanged, i.e., there was no observed, disproportionate increase in these less desired C₁ and C₂ hydrocarbons. These results are therefore indicative of an unexpected increase in the yield of LPG hydrocarbons, arising from the addition of H₂ and CO₂ to synthesis gas containing predominantly H₂ and CO (e.g., having an H₂:CO molar ratio representative of synthesis gas produced by dry reforming and/or steam reforming, such as in a range from about 1.0 to about 3.0, from about 1.0 to about 2.0, or from about 2.0 to about 3.0). Importantly, a convenient source of H₂ and CO₂ useful for this addition is available, according to particular embodiments, as an H₂/CO₂-enriched fraction that may be separated from the effluent of the LPG synthesis reactor and advantageously recycled by combining it with fresh synthesis gas entering the LPG synthesis reactor.

To the extent that the addition of H₂ and CO₂ was observed to cause a reduction in CO conversion, measures to compensate for this offset were investigated. From the standpoint of reaction kinetics, these measures included (a) decreasing throughput through the LPG synthesis reactor (and/or, in an equivalent manner, increasing the reactor size/catalyst weight) to increase reactant residence time and/or (b) increasing the pressure in this reactor to increase reactant concentrations. In terms of a second baseline case for evaluating these measures, the important consideration was the increase in selectivity to LPG hydrocarbons, obtained from FEED C (Example 6), resulting from combined H₂ and CO₂ addition, e.g., which could be realized by operating the process with recycle of a fraction of the effluent from the LPG synthesis reactor, back to this reactor. If maintained at a higher conversion level, this increased selectivity could potentially translate to higher LPG hydrocarbon yields that are very favorable in terms of process economics. To better evaluate these possibilities, two further experiments were performed using compositions of synthesis gas corresponding to FEED C (Example 6), but with (a) a reduced normal flow rate of this feed of 97 ml/min and an increased catalyst weight of 6 grams (Example 7) and (b) additionally an increased LPG synthesis reaction pressure of 3.8 MPa (550 psi) gauge pressure (Example 8). The catalyst bed temperature was maintained at 350° C. (662° F.), and the catalyst composition was unchanged, in terms of having 25 wt-% methanol synthesis catalyst (Cu/ZnO/Al₂O₃), 75 wt-% zeolite beta. Results are summarized in Table 3 below, including CO conversion and percent carbon selectivity for various components of the effluent obtained from the LPG synthesis reactor.

TABLE 3
Variations in residence time/reaction pressure, using FEED C

| | Feed flow rate, ml/min/Mass of MeOH | | |
|---|---|---|---|
| | synthesis catalyst and zeolite beta, g | | |
| | 165/4 | 97/6 | 97/6 |
| Example | 6 | 7 | 8 |
| WHSV, hr⁻¹ | 1.5 | 0.59 | 0.59 |
| Pressure, MPa | 2.1 | 2.1 | 3.8 |
| CO Conversion | 51.6% | 66.1% | 78.3% |
| CH₄ | 13% | 9% | 9% |
| CO₂ | 6% | 21% | 15% |
| ethane | 12% | 9% | 8% |
| propane | 40.0% | 33.7% | 34.1% |
| i-butane | 21.1% | 17.8% | 21.6% |
| n-butane | 6.9% | 8.1% | 9.8% |
| i-pentane | 1% | 1% | 2% |
| n-pentane | 0.0% | 0.0% | 0.0% |
| 2-methyl-pentane | 0.2% | 0.1% | 0.2% |
| 3-methyl-pentane | 0.1% | 0.1% | 0.1% |
| methanol | 0.0% | 0.0% | 0.0% |
| LPG hydrocarbons | 68% | 60% | 66% |
| C3 fraction of LPG | 0.59 | 0.57 | 0.52 |
| LPG Yield | 35% | 39% | 51% |

From a comparison of Examples 6 and 7, CO conversion can be increased by increasing reactant residence time (and/or, in an equivalent manner, reducing throughput or weight hourly space velocity), but not necessarily with a commensurate increase in LPG yield. Rather, depending on other LPG synthesis conditions, including the specific composition of the synthesis gas, increased CO conversion may manifest predominantly in an increase in CO₂ production. Importantly, however, as seen from the results of Example 8, the increase in LPG synthesis reaction pressure allowed the process to operate with a single pass LPG yield exceeding 50%, due to increased conversion with reduced carbon selectivity to CO₂ and increased carbon selectivity to LPG hydrocarbons.

### Ion-Exchange for Preparing a Dehydration Catalyst/Dehydration-Functional Constituent

In preparing ion-exchanged support materials as exemplary components of a dehydration catalyst or dehydration-functional constituent, platinum was selected as a stabilizer and used for exchange with protons (W) initially present in ion-exchange sites of zeolite beta, and more particularly present as BrØnsted acid sites within the zeolite micropores. The zeolite beta had a silica to alumina (SiO₂/Al₂O₃) molar framework ratio of 38, corresponding to an Si/Al molar framework ratio of 19. A solution was prepared of the platinum “precursor,” tetraamine platinum dinitrate, with platinum in this compound having a charge of +2, in view of the two charge-balancing NO₃⁻ ligands. The zeolite beta, either in powder form or in a larger, extrudate form in which the zeolite was bound with an alumina binder, was immersed in this solution for a period to allow near-equilibrium ion-exchange (IE), and excess platinum in the precursor solution was thereafter washed from the support material. Even after ion-exchange, the +2 valency of Pt could be partially satisfied by the salt ligands, meaning that a single Al⁺³ ion-exchange site could be associated ionically with a Pt-nitrate group, having the nitrate ligand still attached and thereby providing a charge of +1 with respect to the group as a whole. Following subsequent calcination that removed the nitrate ligands, the +2 valency of Pt could still be at least partially satisfied by oxygen bonds, such as bonds within the silica structure, bonds with hydroxyl groups, etc. As a practical matter, therefore, the Pt:exchange site molar ratio or stoichiometry could be as high as 1:1, but this could also vary, for example, from 1:3 to 1:1, or from 1:2 to 1:1, depending on the precursor used and corresponding valency of the platinum or other stabilizer.

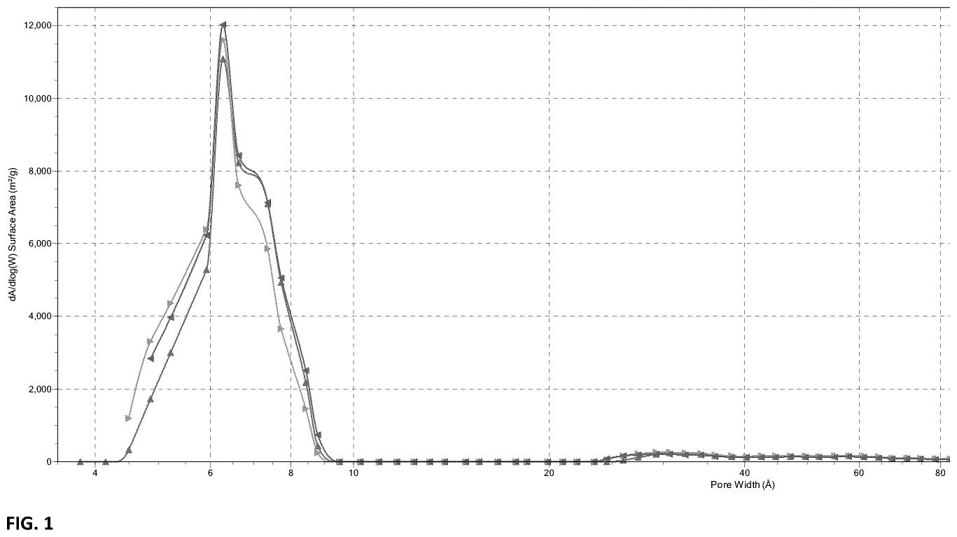

Nitrogen (N₂) physisorption with density functional theory (DFT) fitting, i.e., “N₂-DFT,” is a technique used to characterize the pore size distributions of materials in the micropore range (pores smaller than 2 nm in diameter). This is a pore size range in which other common techniques, such as the Barrett-Joyner-Halenda (BJH) model, do not work. According to the N₂-DFT results provided in FIG. 1 (“Pt IE” vs. “No Pt”), zeolite beta having been ion-exchanged with platinum as described above has slightly lower volume of micropores in the 6-7 Angstrom range but is otherwise structurally the same as the zeolite beta in its initial hydrogen form. That is, the protons, or H⁺ ions, in the micropores of the support material were exchanged with larger Pt ions (or Pt⁺ groups) without otherwise affecting its physical structure. Such Pt⁺ groups can be more generally represented as “Pt^{δ+},” with the delta symbol indicating some variance about the stated charge of +1.

For comparative purposes, a sample of zeolite beta, including the same amount of platinum as incorporated by ion-exchange, was prepared instead by incorporating this stabilizer using incipient wetness impregnation (IWI). This comparative sample was likewise characterized by the N₂-DFT method. From the results provided in FIG. 1 (“Pt IWI” vs. “Pt IE”), with respect to the sample Pt IWI, although some Pt was deposited in the 6-7 Angstrom pores as evidenced by a lowering of the magnitude of the surface area peak in this range, there was a general broad loss of pore volume throughout the micropore range up to about 9 Angstroms and beyond. In contrast to the IE method, which more selectively deposits Pt in ion-exchange sites as opposed to other areas, the IWI method deposited Pt throughout the zeolite micropores. In contrast to IE, which produced, with greater consistently, more specific active sites in specific coordination environments, IWI produced heterogeneous active sites that could unselectively promote side reactions.

Following the ion-exchange technique described above, a series of platinum-exchanged zeolite beta support materials, for dehydration catalysts or dehydration-functional constituents, were prepared using precursor solutions having differing concentrations of platinum. This resulted in correspondingly different degrees of ion-exchange, meaning that the support material had different amounts, or weight percentages, of platinum incorporated by ion-exchange. More particularly, dehydration catalysts were obtained, comprising zeolite beta and having amounts of ion-exchanged platinum, as indicated in Table 4 below. These amounts were determined by Inductively Coupled Plasma (ICP) spectroscopy.

TABLE 4
Preparations of Pt-exchanged zeolite beta dehydration catalysts

| | wt-% Pt in the final formulated catalyst |
|---|---|
| Catalyst 1 | 0.04% |
| Catalyst 2 | 1.07% |
| Catalyst 3 | 1.48% |
| Catalyst 4 | 2.00% |

### Performance Evaluation of Ion-Exchanged Support Material

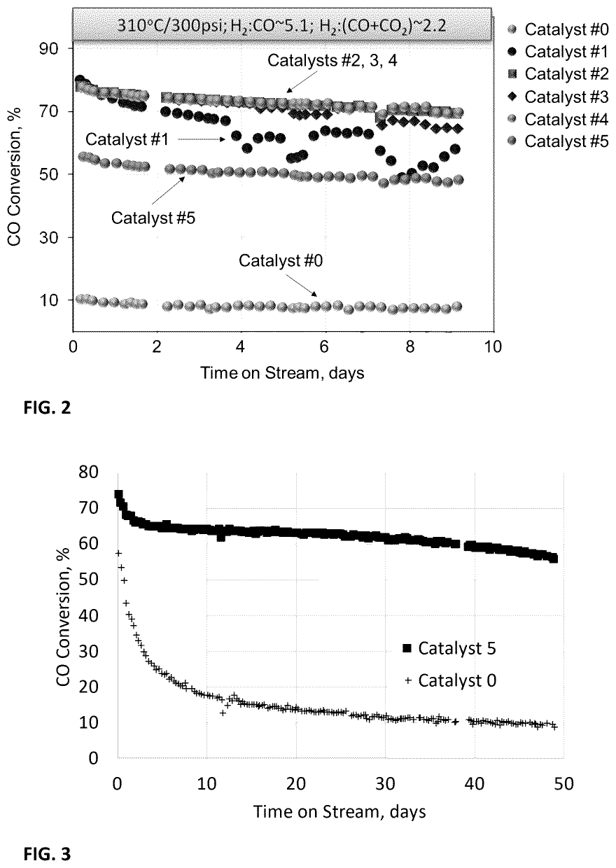

LPG synthesis catalyst systems including a methanol synthesis catalyst (Cu/ZnO/Al₂O₃), together with each of the platinum-exchanged zeolite beta preparations as dehydration catalysts, according to CATALYST 1, CATALYST 2, CATALYST 3, and CATALYST 4, as shown in Table 4 above, were tested for their activities to convert a 5:1 H₂:CO molar ratio, and 2.2 H₂:(CO+CO₂) molar ratio, synthesis gas. Another dehydration catalyst, namely CATALYST 5, prepared from zeolite beta in powder form with approximately the same ion-exchanged Pt stabilizer loading as used for CATALYST 2, was likewise tested in this manner. The LPG synthesis catalyst system was prepared with 40 wt-% of the methanol synthesis catalyst and 60 wt-% of the zeolite beta. Other LPG synthesis conditions included 2.1 MPa (300 psig) gauge pressure and 310° C. (590° F.) catalyst bed temperature. The synthesis gas feed was 13 mol-% CO, 17 mol-% CO_{2,} 67 mol-% H₂, and 3 mol-% N₂, which was fed to the LPG synthesis catalyst system at 70 normal milliliters/min. According to the results in FIG. 2, at an amount of at least about 1 wt-% ion-exchanged Pt, the dehydration catalyst and LPG synthesis catalyst system as a whole exhibited a favorable activity in terms of CO conversion and a high level of stability. Less favorable activity and stability characteristics were observed (a) in the case of the Pt stabilizer being absent, namely with respect to CATALYST 0, and (b) in the case of only a small amount of ion-exchanged platinum stabilizer, namely with respect to CATALYST 1. In FIG. 2, the CO conversion data for both CATALYST 0 and CATALYST 5 are shown following an on-stream operating period of about 50 days, i.e., following their use in obtaining the results shown in FIG. 3.

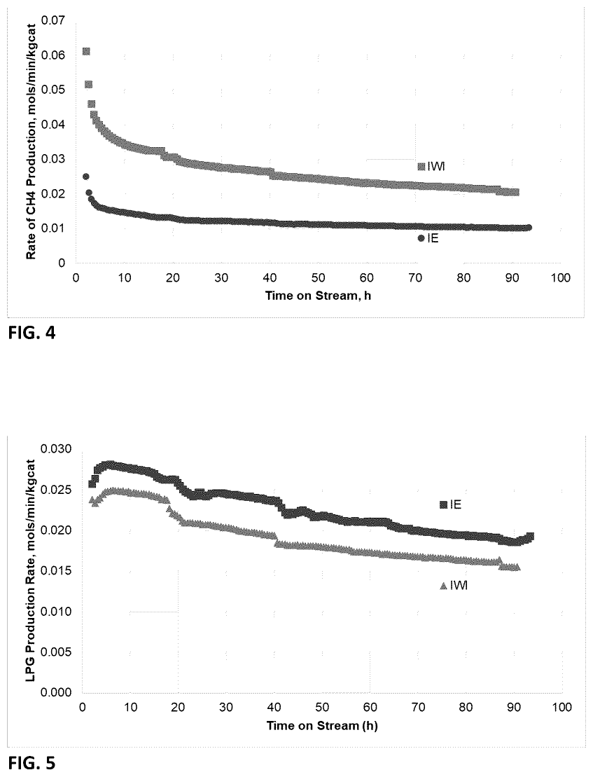

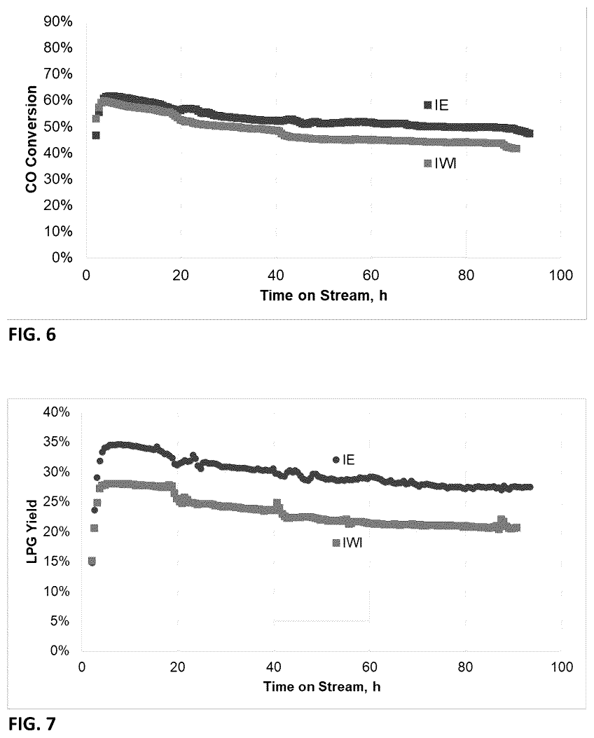

Further tests were conducted, under the same conditions and with the same synthesis gas feed, to compare the performance of zeolite beta preparations as dehydration catalysts, in which the same amount of platinum stabilizer, namely approximately 1 wt-%, was loaded by ion-exchange (IE) versus incipient wetness impregnation (IWI). The comparative performance results are shown FIGS. 4-7. Whereas both catalysts exhibited similar CO conversion percentages over time, as shown in FIG. 6, it can be appreciated from FIG. 4 that the dehydration catalyst prepared by IWI (“IWI catalyst”) converted a greater proportion of CO unselectively to methane (in moles per minute per kilogram of catalyst), compared to the dehydration catalyst prepared by IE (“IE catalyst”). This corresponded to a lower rate of LPG hydrocarbon production (in moles per minute per kilogram of catalyst) for the IWI catalyst, compared to the IE catalyst, as shown in FIG. 5, in addition to a lower LPG hydrocarbon yield, as shown in FIG. 7. Without being bound by theory, it is believed that Pt particles (including nanoparticles) loaded onto support materials by deposition techniques (such as IWI) have a certain activity for CO and CO₂ methanation, and this catalytic function can be suppressed when the catalyst is prepared by IE. This may be due to the Pt in the zeolite prepared by IE being “tethered” to specific coordination environments during ion-exchange, thereby producing a homogeneous set of active sites to promote the desired LPG synthesis conversion pathways. The stability of catalysts made according to either preparation technique was similar. Likely, during IWI, some Pt is deposited in ion-exchange sites within the catalyst pores, as well as in other environments, as evidenced by the N₂ physisorption data (N₂-DFT). Therefore, some Pt is likely still present in the ion-exchange sites within the catalyst micropores of the IWI sample, promoting activity and stability. However, IE can result in a more precise distribution of Pt, to better promote stability and activity, without promoting undesirable side-reactions such as methane formation.

Overall, aspects of the invention relate to LPG synthesis catalyst systems that provide activities for both alcohol (e.g., methanol) synthesis and in situ dehydration of the alcohol (e.g., methanol) to hydrocarbons, and particularly the LPG hydrocarbons propane and/or butane. These catalyst systems benefit from the incorporation of a stabilizer such as platinum and/or yttrium (e.g., as yttria or yttrium oxide) and/or promoters such as manganese (Mn), magnesium (Mg), and/or silicon (Si) into these catalyst systems, to improve performance characteristics such as activity and/or stability, as well as selectivity to, and/or yield of, desired LPG hydrocarbons. Those skilled in the art having knowledge of the present disclosure, will recognize that various changes can be made to LPG synthesis catalyst systems and associated processes, in attaining these and other advantages, without departing from the scope of the present disclosure. As such, it should be understood that the features of the disclosure are susceptible to modifications and/or substitutions without departing from the scope of this disclosure. The specific embodiments illustrated and described herein are for illustrative purposes only, and not limiting of the invention as set forth in the appended claims.

1. An LPG synthesis catalyst system comprising:
(i) an alcohol synthesis catalyst, and
(ii) a dehydration catalyst,
wherein the catalyst system comprises a stabilizer that reduces deactivation of the dehydration catalyst.

2. The LPG synthesis catalyst system of claim 1, wherein the alcohol synthesis catalyst is a methanol synthesis catalyst.

3. The LPG synthesis catalyst system of claim 1, wherein the stabilizer is a noble metal stabilizer or a non-noble metal stabilizer, said non-noble metal stabilizer being in elemental form or a compound form.

4. The LPG synthesis catalyst system of claim 3, wherein the noble metal stabilizer is platinum.

5. The LPG synthesis catalyst system of claim 3, wherein the non-noble metal stabilizer is a metal selected from Group 3 or Group 4 of the Periodic Table, or a lanthanide.

6. The LPG synthesis catalyst of claim 4, wherein the non-noble metal stabilizer is yttrium (Y).

7. The LPG synthesis catalyst system of claim 1, wherein (i) and (ii) are separate compositions, each composition being in the form of separate particles.

8. The LPG synthesis catalyst system of claim 1, wherein (i) and (ii) are present in the catalyst system in a weight ratio of (i):(ii) from about to about 1:10.

9. The LPG synthesis catalyst system of claim 3, wherein the non-noble metal stabilizer is in the compound form, said compound form being an oxide or a carbonate.

10-25. (canceled)

26. An LPG synthesis catalyst system comprising, as constituents of a bi-functional catalyst:
(i) an alcohol synthesis-functional constituent, and
(ii) a dehydration-functional constituent,
wherein the catalyst system comprises a stabilizer that reduces deactivation of the LPG synthesis catalyst system.

27. The LPG synthesis catalyst system of claim 26, wherein the alcohol synthesis-functional constituent is a methanol synthesis-functional constituent.

28. The LPG synthesis catalyst system of claim 27, wherein the stabilizer is a noble metal stabilizer or a non-noble metal stabilizer, said non-noble metal stabilizer being in elemental form or a compound form.

29. The LPG synthesis catalyst system of claim 28, wherein the noble metal stabilizer is platinum.

30. The LPG synthesis catalyst system of claim 28, wherein the non-noble metal stabilizer is a metal selected from Group 3 or Group 4 of the Periodic Table, or a lanthanide.

31. The LPG synthesis catalyst system of claim 30, wherein the non-noble metal stabilizer is yttrium (Y).

32-37. (canceled)

38. An alcohol to LPG hydrocarbon conversion catalyst comprising a stabilizer on a solid acid support comprising a zeolite or a non-zeolitic molecular sieve, wherein the stabilizer reduces deactivation of the alcohol to LPG hydrocarbon conversion catalyst.

39. The alcohol to LPG hydrocarbon conversion catalyst of claim 38, which is a methanol to LPG hydrocarbon conversion catalyst.

40. The alcohol to LPG hydrocarbon conversion catalyst of claim 38, wherein the stabilizer is a noble metal stabilizer or a non-noble metal stabilizer, said non-noble metal stabilizer being in elemental form or a compound form.

41. The alcohol to LPG hydrocarbon conversion catalyst of claim 38, wherein the stabilizer is present in ion-exchange sites of the zeolite or non-zeolitic molecular sieve.

42. The alcohol to LPG hydrocarbon conversion catalyst of claim 40, wherein the noble metal stabilizer is platinum.

43-46. (canceled)
